(19) United States
(12) Patent Application Publication
(10) Pub. No.: US 2020/0149065 A1
(43) Pub. Date: May 14, 2020

(54) FOAMY VIRAL VECTOR COMPOSITIONS AND METHODS FOR THE MANUFACTURE OF SAME

(71) Applicant: Children's Hospital Medical Center, Cincinnati, OH (US)

(72) Inventors: Johannes C.M. van der Loo, Newtown Square, PA (US); David William Russell, Seattle, WA (US); Punam Malik, Cincinnati, OH (US); Md Nasimuzzaman, Cincinnati, OH (US)

(21) Appl. No.: 16/562,578

(22) Filed: Sep. 6, 2019

Related U.S. Application Data
(63) Continuation of application No. 15/041,087, filed on Feb. 11, 2016, now abandoned.
(60) Provisional application No. 62/127,956, filed on Mar. 4, 2015.

(51) Int. Cl. C12N 15/86; C12N 7/00

(52) U.S. Cl. CPC C12N 15/86; C12N 2740/17043; C12N 2740/17051; C12N 7/00

(57) ABSTRACT

Disclosed are methods of preparing FV vector particles. In some aspects, the disclosed methods may include the steps of transfecting a population of eukaryotic cells by contacting said population of eukaryotic cells with one or more transfection reagents to form a transfection mixture, and incubating the transfection mixture to form a transfected cell population; harvesting the FV vector particles from said transfected cell population; purifying the FV vector particles; and concentrating the FV vector particles.

### CROSS REFERENCE TO RELATED APPLICATION

This application is a continuation of U.S. patent application Ser. No. 15/041,087 entitled “Foamy Viral Vector Compositions and Methods for the Manufacture of Same,” filed Feb. 11, 2016, now abandoned, which claims the benefit of and priority to U.S. Ser. No. 62/127,956, filed Mar. 4, 2015, of same title, in its entirety for all purposes.

### GOVERNMENT RIGHTS

This invention was made with government support under HL070871, HL085107, and TR000077 awarded by NIH. The government has certain rights in the invention.

### BACKGROUND OF THE INVENTION

Foamy virus (FV) vectors are a promising alternative to gamma-retroviral and lentiviral vectors, demonstrating high transduction rates (1, 2) with less genotoxicity (3, 4). Additional advantages include the fact that the FV envelope has tropism for most cell types, the vector can carry larger expression cassettes as compared to gamma-retroviral and lentiviral vectors, the vector particles have increased stability due to a DNA genome formed in developing vector particles as compared to RNA, and FV is not associated with disease in humans (5). Combined, these properties make FV vector system the ideal candidate for gene therapy application. Proof of principle on the use of FV vectors for genetic correction was provided by Bauer et al. (6, 7) who demonstrated cure of dogs suffering from canine Leukocyte Adhesion Deficiency (LAD). The study used autologous CD34+ cells transduced with FV vector carrying the CD18 gene driven by the Murine Stem Cell Virus (MSCV) promoter. In 4-7 years of follow-up, there has been no emergence of clonal dominance or leukemia, supporting the claim that FV vectors are safe for clinical application.

To date, there has not been a concerted effort, published or unpublished, to generate, purify and highly concentrate FV vector particles for clinical application. Given the advantageous properties of FV, the ability to increase the titer of FV translates into practical benefits. Thus, there is a need in the art for improving titer of FV and methods for large-scale production of FV. The instant application addresses one or more such needs in the art.

### BRIEF SUMMARY

Disclosed herein are methods of preparing FV vector particles, particularly to increase titer. The methods may comprise, in some aspects, the steps of transfecting a population of eukaryotic cells by contacting the population of eukaryotic cells with one or more transfection reagents to form a transfection mixture, and incubating the transfection mixture to form a transfected cell population; harvesting the FV vector particles from the transfected cell population, wherein the harvesting step may be carried out about 70 hours to about 100 hours, or about 70 hours to about 90 hours, or about 70 hours to about 80 hours, or about 72 hours to about 75 hours, post-transfection; purifying the FV vector particles; and concentrating the FV vector particles.

### BRIEF DESCRIPTION OF THE DRAWINGS

FIG. 1 depicts a graph showing that optimal titer of FV vector requires the presence of 10% Fetal Bovine Serum (FBS). Transfection of 293T with FV-GFP using Calcium phosphate. Titer on HT1080 (Avg±SD, duplicate).

FIG. 2 depicts a graph showing that different lots of Fetal Bovine Serum (FBS) generate a different amount of FV vector transfection of 293T with FV-GFP using Calcium phosphate. Titer on HT1080 (Avg±SD, triplicate).

FIG. 3 depicts a graph showing the effect of a lipid supplement (Gibco Chemically-Defined Lipid Concentrate, Catalog #11905-031) in serum-free and low-serum media (D2, DMEM with 2% FBS; D10, DMEM with 10% FBS). Results show that DMEM with 10% FBS is superior (Avg±SD, duplicate).

FIG. 4 depicts a graph showing the effect of concentration of DMSO on the recovery of infectious FV vector after storage at −80° C. This data demonstrates that reduction in the amount of DMSO reduces the recovery of infectious virus.

FIG. 5 depicts a graph showing a comparison of the standard Calcium Phosphate (CaPhos) transfection method vs. PolyPlus “PEIPro” Transfection Reagent at various amounts (25 to 80 microLiter) per 10 cm dish, using a Foamy GFP vector. The data show increased titer with PEI as compared to Calcium Phosphate (Avg±SD, triplicate).

FIG. 6 depicts a graph showing a comparison of FV vector titer derived from transfection of 293T cells with calcium phosphate and PEI (PolyPlus PEIPro) at different amounts (volume PEI used per 10 cm tissue culture dish equivalent). Volume of 70 microLiter PEI per plate is optimal (Avg±SD, duplicate).

FIG. 7 depicts a graph showing a comparison of two methods of adding PEI compatible with large scale manufacturing: (1) mixing of cells with PEI and plasmid prior to plating; and (2) mixing of PEI and plasmid with media added to adherent cells. Controls are calcium phosphate and PEI using lab-scale methods. Results show that mixing PEI with media but not cells provides results similar to the Optimal PEI method (Avg±SD, triplicate).

FIG. 8 depicts a graph showing a comparison of complexion time of plasmid and PEI showed 10 minutes to be optimal as compared to 15 or 20 minutes. Data show titer of FV-GFP (Avg±SD, triplicate).

FIG. 9 depicts a graph showing a comparison of plates pre-seeded 3 days prior to transfection with PEI vs. the standard 1 day, using either existing media or fresh media for transfection, showed plating of 1 day prior to PEI transfection with fresh media to be optimal. Titers FV-GFP (Avg±SD, triplicate).

FIG. 10 depicts a graph showing the effect of different concentrations of optimized Gag plasmid (pCiGAGopt) on the titer of FV vector as compared to the standard amount (10.4 microgram) of non-optimized Gag (pCIGSΔΨ). Data show a 5-fold improvement in titer using 16-fold diluted pCiGAGopt. Titers FV-CD18 (Avg±SD, duplicate).

FIG. 11 depicts a graph showing FV Titer generated by transfection with PEI without and with media change 19 hours post-transfection. The data show that more FV vector is produced when the media is not changed the morning after transfection (Avg±SD, triplicate).

FIG. 12 depicts a graph showing FV Titer generated by transfection with PEI with and without media change the day post-transfection, with and without treatment of the plastic with Poly-L-Lysine (PLL). The data show that more FV vector is produced when the media is not changed the morning after transfection, using PLL-coated plates. FV-GFP (Avg±SD, triplicate).

FIG. 13 depicts a graph showing evaluation of the pattern of FV-CD18 virus production when no media was changed after transfection. Infectious titers were measured on Raw 264.7 cells (Avg±SD, triplicate). The data show that virus titers are the highest at 66 hours post-transfection.

FIG. 14 depicts a graph showing evaluation of the elution profile of FV-GFP using a salt gradient from 150 mM to 1 M NaCl after binding to a POROS-Heparin column. Data show that infectious virus eluted between 190 and 587 mM NaCl (Conductivity of 22-52 mS/cm). Based on this, the salt concentration for step elution on the AKTAReady was set at 600 mM NaCl.

FIG. 15 shows that 705 mL of FV-CD18 vector can be effectively loaded onto a 7.9 mL POROS-Heparin (at 267 cm/h with a 2.3 min residence time) without breakthrough, and eluted using 600 mM NaCl buffer with a recovery of infectious virus of 75%. The graph (top) shows the loading volume (Red Line) versus recovery of infectious FV-CD18 virus as measured on Raw 264.7 cells (Blue Curve). Chromatogram (bottom).

FIGS. 16A-16C depict a graph and related data showing the concentration (20-fold) of POROS-Heparin purified FV-CD18 vector using TFF. FIG. 16A depicts a graph showing data demonstrating stable pressures without evidence of membrane fouling, using an average Trans-Membrane Pressure (TMP) of 2 psi. FIG. 16B depicts the figure legend for FIG. 16A. FIG. 16C depicts the results at a flux rate of 50 LMH.

FIG. 17 depicts photographs showing ultracentrifugation (19,000 RPM, 11° C., 2 hours) and pelleting of POROS-Heparin purified and Tangential Flow Filtration (TFF)-concentrated FV-CD18 vector using capped Optiseal BellTop Polyallomer tubes (Beckman Coulter; top pictures), blunt needle for supernate removal (bottom-left), and an extended long-range pipette tip (bottom-right) for re-suspension of the pellet and retrieval of the vector product.

FIG. 18 depicts a graph showing the effect of benzonase treatment on recovery of FV-GFP vector. Benzonase was added at the media change step post-transfection at concentrations ranging from 0 to 200 U/mL in the presence of 10 mM MgCl₂. The data show that FV production is not negatively affected by benzonase up to 200 U/mL for 16 hours and that benzonase can be safely used to reduce residual plasmid during production or after harvest (Avg±SD, triplicate).

FIG. 19 depicts a graph showing the effect of benzonase on the recovery of FV-GFP. Benzonase was added at 50 U/mL to cells post-transfection in the presence of 10 mM MgCl₂ and incubated for 16 or 40 hours (Avg±SD, triplicate). The data demonstrates that longer incubation with benzonase does not negatively impact FV titer and that benzonase treatment can be extended to 40 hours if needed.

FIG. 20 depicts a graph showing that 40 million 293T cells per T225 (or 1.8×10⁵ cells/cm²), from a range of 20 to 50 million cells per T225, is the optimal density at the time of transfection for production of FV-GFP (Avg±SD, triplicate).

FIG. 21 depicts a graph showing the evaluation of amount of FV-CD18 in tissue culture flasks supplemented with 10% or 20% fresh DMEM media with FBS at 48 hours post-transfection (Avg±SD, triplicate) to evaluate if fragility of the adherent cell layer and titers could be improved. The data show that addition of fresh media did not impact the cell layer or titer and that fragility of the cell layer is not due to nutrient depletion.

FIG. 22 depicts a graph showing that after Tangential-Flow Filtration (TFF), FV-CD18 can be filtered through a 0.8 micron filter without loss of titer, and through a 0.45 and 0.22 micron filter with minimal loss of titer showing a 94% and 84% recovery of infectious particles, respectively.

FIG. 23 depicts a graph showing a stability study of FV-GFP non-purified vector supernate shows that FV is stable for at least 3 days at 4° C. supporting purification to take place over several days with minimal loss of infectious titer. Titered in triplicate (±SE).

FIG. 24 depicts a graph showing that incubation of POROS-Heparin purified FV-GFP with NaCl at ambient temperature for 2 hours shows a loss of infectious titer with increasing NaCl concentration. This supports the notion that upon elution from the chromatography column with approximately 600 mM NaCl, product needs to be diluted to approximately 150 mM NaCl as soon as possible to preserve titer.

FIG. 25 depicts a graph showing that titer of FV vector to different concentrations of NaCl for 15 minutes, followed by dilution to 150 mM NaCl prior to storage (corrected for the dilution factor). Titer is significantly reduced by higher molarity of NaCl concentration-dependent (Avg±SD, triplicate). These data supports that FV eluted from POROS-Heparin with 600 mM NaCl should be diluted as soon as possible to isotonic conditions.

FIG. 26 depicts a graph showing that incubation of FV-GFP at pH 6.9 to 10.1 for 0-24 hours shows loss of infectious titer above pH 8.

FIG. 27 depicts a graph showing the results of experiments in which POROS-Heparin purified FV-GFP stored frozen at −80° C. in the presence of 5% DMSO was thawed, maintained at ambient temperature for 1, 2 or 3 hours, and re-frozen (Avg±SD, triplicate). Data show no loss of titer indicating that final vector product can be thawed, pooled and aliquoted without significant loss of titer.

FIG. 28 depicts a graph showing that storage of FV samples at −80° C. in the presence of 300 mM NaCl or higher and 5% DMSO rendered FV completely and irreversibly non-infectious. Percentage GFP of HT1080 cells transduced with samples derived from a POROS-Heparin chromatography run. Cells were transduced with samples fresh or after frozen storage in presence of 5% DMSO. This illustrates that FV vector cannot be stored frozen in the presence of DMSO unless in an isotonic media at 150 mM NaCl.

FIGS. 29A-29F depict the optimization of FV vector transfection to maximize FV vector titers. HEK293T cells were transfected under various experimental conditions with FV vector plasmids. FV vector supernatants were harvested three days posttransfection and titer (IU/ml) was estimated by infecting HT1080 cells (FV-GFP) or RAW264.7 cells (FV-hCD18). FIG. 29A depicts FV-GFP plasmid transfection using calcium phosphate or increasing concentrations of PEI, ranging from 25 to 80 μg per T75 flask (n=3, *P≤0.05, as compared to CaPO4 transfection). FIG. 29B depicts FV-hCD18 plasmid transfection using increasing concentrations of PEI (n=3, *P≤0.05, as compared to 40 μg PEI). FIG. 29C depicts FV-GFP plasmid transfection in culture vessels untreated (left bar) or treated (right bar) with poly-1-lysine (n=3, *P≤0.05). FIG. 29D depicts FV-GFP plasmid transfection using various PEI-DNA precipitation time (n=3, *P≤0.05, as compared to a 15 minute precipitation time). FIG. 29E depicts FV-GFP plasmid transfection with or without change of PEI containing transfection media from producer cells (n=3, *P≤0.05). FIG. 29F depicts Optimization of harvest time after FV-hCD18 plasmid transfection (n=3, *P≤0.05, as compared to the 24-hour time point).

FIG. 30 depicts optimization of FV vector transfection to maximize FV vector titers using two transfection methods and different amounts of PEI. HEK293T cells were transfected with FV-GFP vector plasmids using increasing concentrations of PEI, ranging from 60-100 μg per T75 flask. FV vector supernatants were harvested three days post-transfection and titer (IU/mL) was estimated by infecting HT1080 cells (n=3, *P≤0.05, as compared to 60 μg PEI).

FIGS. 31A and 31B depict the use of codon-optimized gag plasmid for FV vector production. FV-hCD18 vectors were produced by PEI-mediated transfection of HEK293T cells with FV vector packaging plasmids, including either the previously optimized amount of the original gag plasmid (pCiGS 10.4 μg) or various amounts of the codon-optimized gag plasmid (pCiGAGopt). Vector supernatants were harvested three days post-transfection and titers were estimated using RAW 264.7 cells. FIG. 31A depicts transfection of HEK293T cells with amounts of pCiGAGopt ranging from 10.4 to 1.3 μg per T75 flask (n=3, *P≤0.05, as compared to 10.4 μg of pCiGSΔΨ). FIG. 31B depicts transfection of HEK293T cells with amounts of pCiGAGopt ranging from 2.6 to 0.16 μg per T75 flask (n=3, *P≤0.05, as compared to 10.4 μs of pCiGSΔΨ).

FIGS. 32A and 32B show benzonase treatment of FV vector supernatants. Benzonase was added to producer cell cultures for 16 hours. FV-GFP vector supernatants were harvested three days post-transfection and titers were estimated using HT1080 cells. FIG. 32A depicts treatment of FV vectors with 50, 100 or 150 U/mL of Benzonase (n=3, P≥0.05). FIG. 32B depicts optimization of timing of Benzonase treatment (50 U/mL) 16 or 40 hours prior to harvest of FV vector (n=3, P≥0.05). Control group does not contain Benzonase.

FIG. 33 shows purification of FV-GFP vector supernatants with heparin affinity chromatography. Nuclease-treated FV-GFP vector supernatant was loaded onto a POROS heparin column and washed with buffer containing 150 mM NaCl. The heparin bound FV particles were eluted using a continuous NaCl gradient. The optimal NaCl concentration for elution of FV vector was estimated based on vector titer and conductivity in each fraction. Line with grey squares shows the conductivity; line with dark circles shows the total infectious FV-GFP vector particles.

FIG. 34 depicts purification of FV-hCD18 vector supernatants with heparin affinity chromatography. FV-hCD18 vectors were purified using optimal conditions of sample loading and washing. The elution was carried out with 600 mmol/1 NaCl. Infectious unit (IU) of FV vectors was estimated after infecting cells with the diluted fractions of FV vector samples. Line with gray squares shows the volume of FV sample loaded; the line with dark diamonds shows the total infectious units of FV-hCD18 vector in each fraction (43.5 ml).

FIG. 35 shows the effect of dimethyl sulfoxide (DMSO) treatment on the viability of human CD34+ cells. Human CD34+ cells derived from mobilized peripheral blood (MPB) were cultured in X-VIVO 10 medium supplemented with human cytokines SCF (300 ng/mL), Flt3L (300 ng/mL) and TPO (100 ng/mL) along with indicated concentration of DMSO. The trypan blue dye-exclusion method was used to assess cell viability. Y-axis shows the percentages of viable cells. The duration of treatments is indicated. Statistical analysis was done using Dunnett's multiple comparison ANOVA, with all groups compared to 0% DMSO. Statistically significant groups (P<0.05) are indicated: ** P<0.01 and **** P<0.0001.

FIG. 36 depicts Flow diagram of FV vector production and purification. HEK293T producer cells were seeded in cell culture vessels treated with poly-1-Lysine and FV vector plasmids were transfected into the cells with PEI. Cultures were treated with Benzonase for 16 hours prior to vector harvest. FV supernate was filtered, purified with heparin affinity chromatography and filtered, concentrated, and diafiltered with TFF, sterile filtered, and concentrated aseptically using ultracentrifugation. FV vector supernatants were stored at −80 ÅãC in the presence of 5% DMSO.

FIG. 37 depicts a schematic diagrams of the FV vector plasmids. (a) pΔϕ-MSCV-GFP, FV-GFP gene transfer vector plasmids, (b) pΔϕ-MSCV-huCD18, FV-hCD18 gene transfer vector plasmids, (c) pCiGSΔΨ, FV gag gene packaging plasmid, (d) pCiPS, FV pol gene packaging plasmid, (e) pCiES, FV env gene packaging plasmid. CMV, cytomegalovirus; LTR, long terminal repeat; MSCV, murine stem cell virus LTR promoter; GFP, green fluorescent protein; hCD18, human CD18 cDNA; SV40 SD/SA, SV40 splice donor and acceptor.

FIGS. 38A and 38B depict titration of hCD18 vector. Titer of FV-hCD18 was determined by transducing mouse RAW264.7 cells. Transduced cells were identified using a fluorescently labeled mouse anti-human CD18 monoclonal antibody. Flow cytometry plots for Untransduced cells are depicted in FIG. 38A, FIG. 38B depicts cells transduced with FV-hCD18 vector.

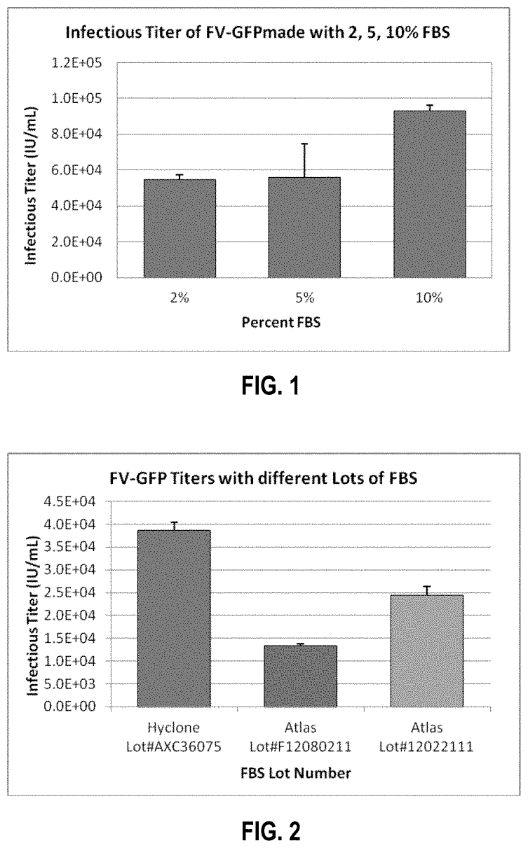

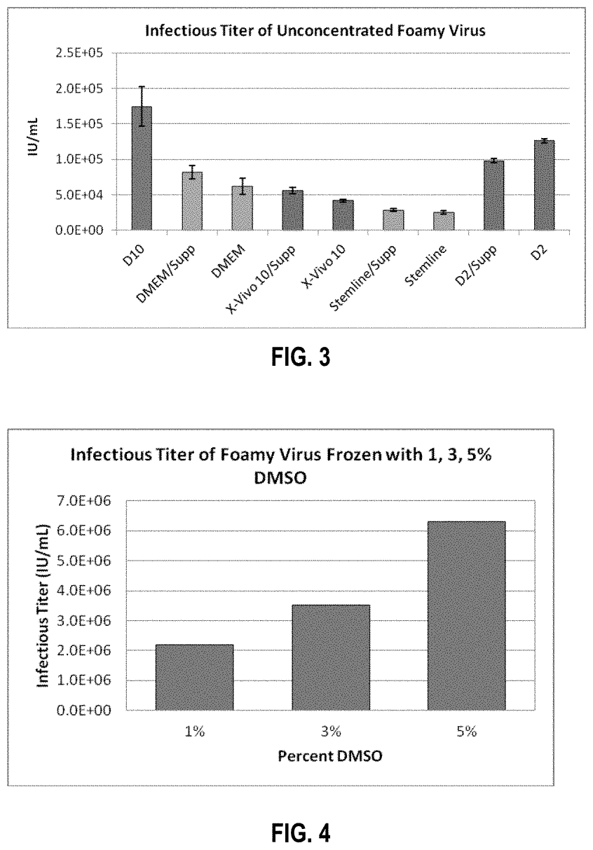

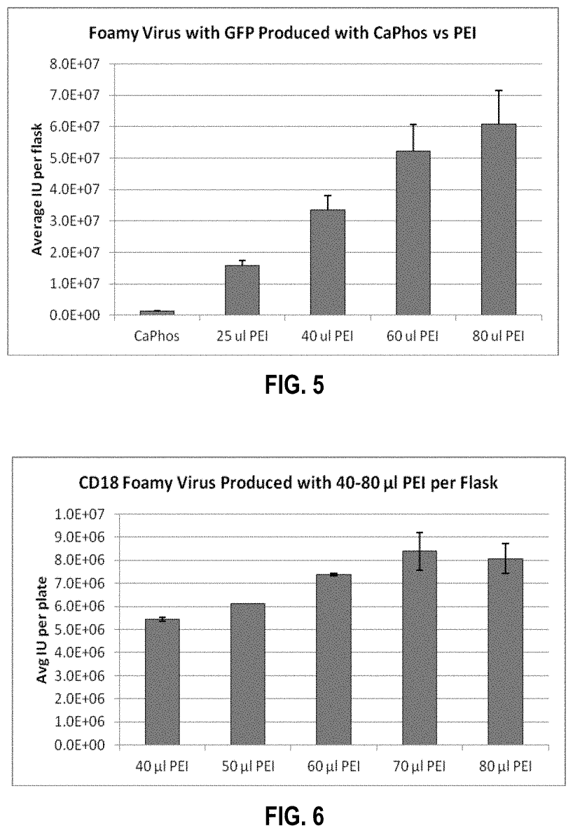

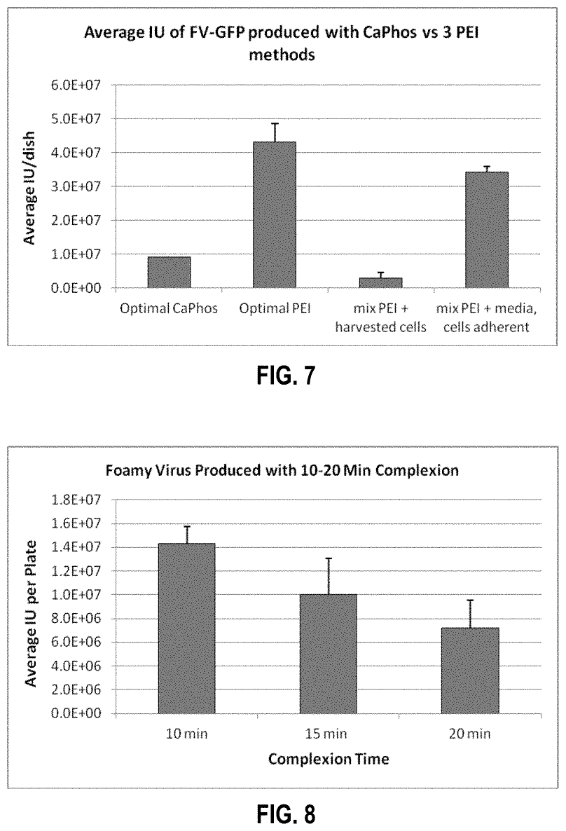

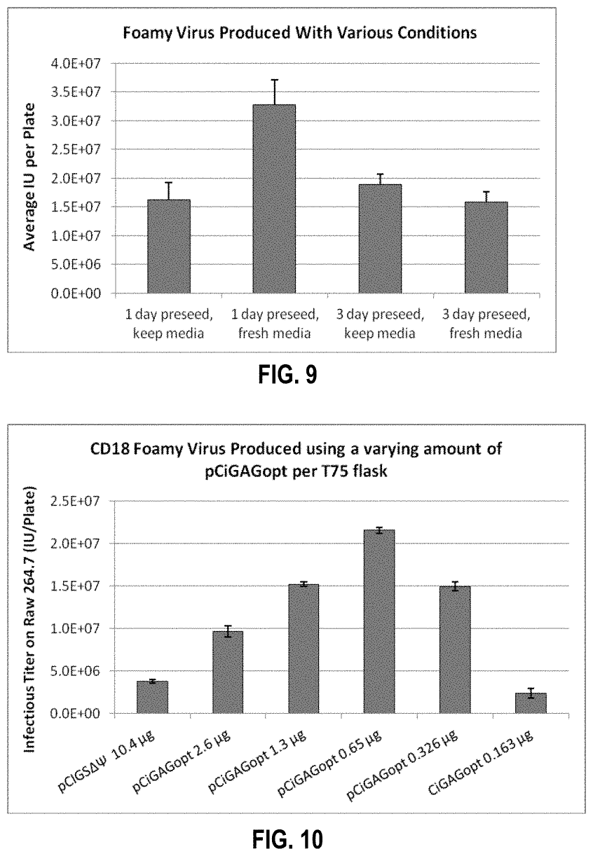

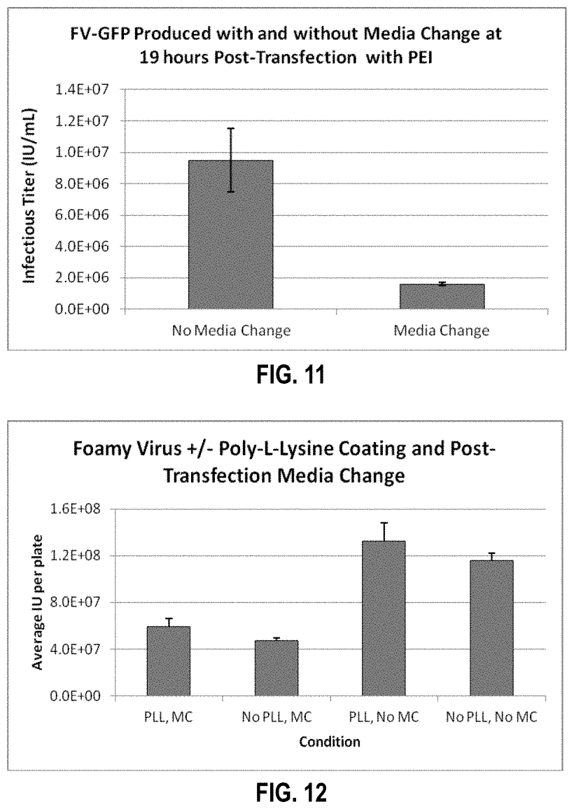

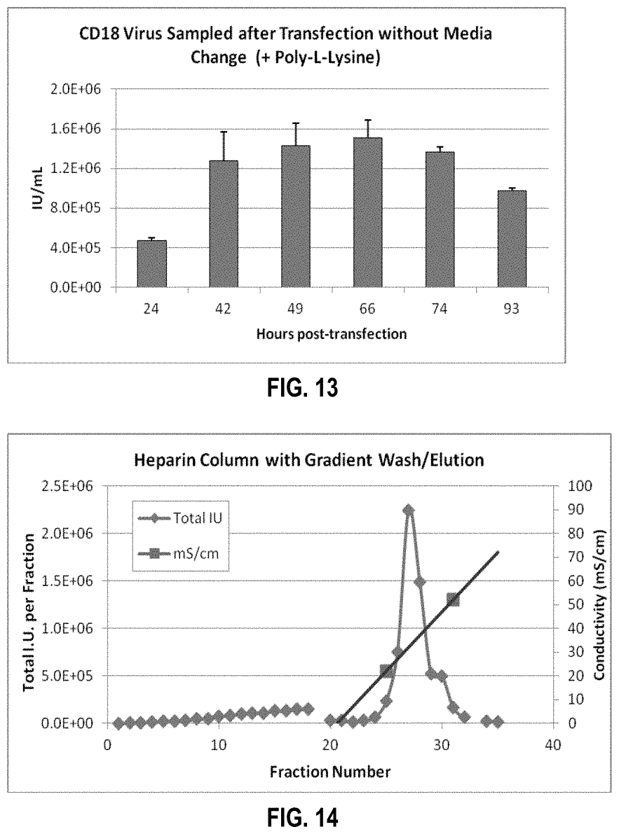

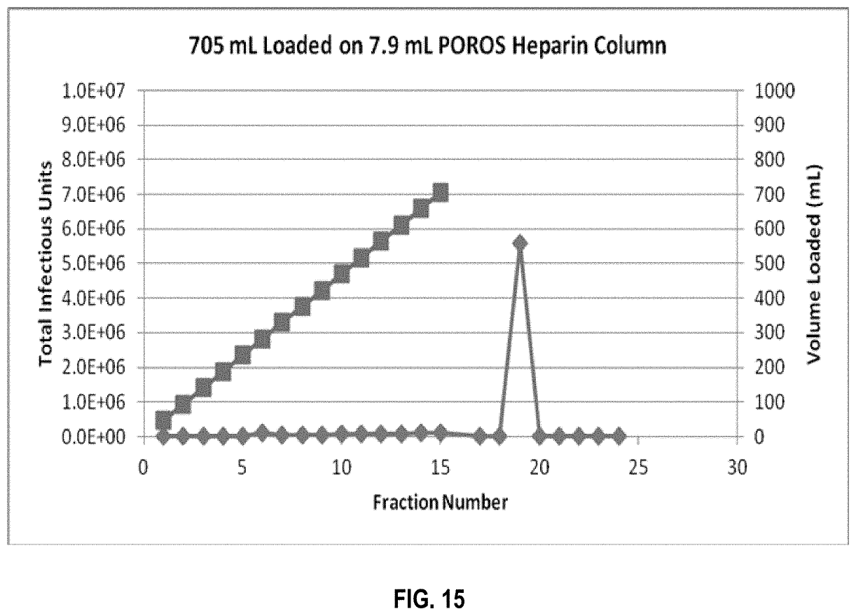

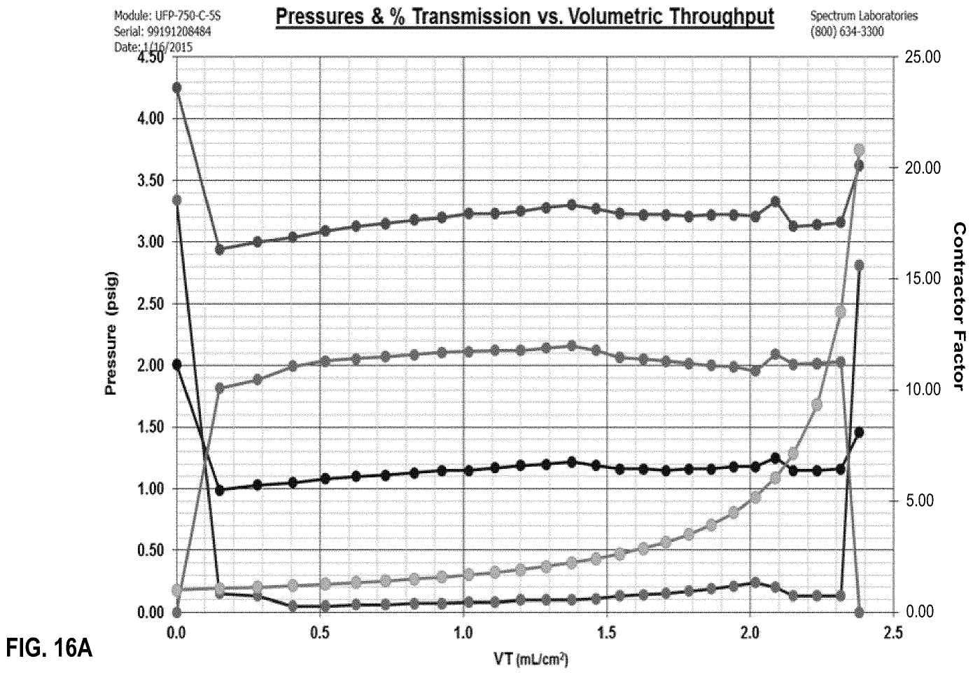

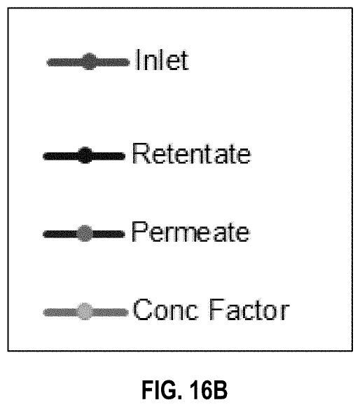

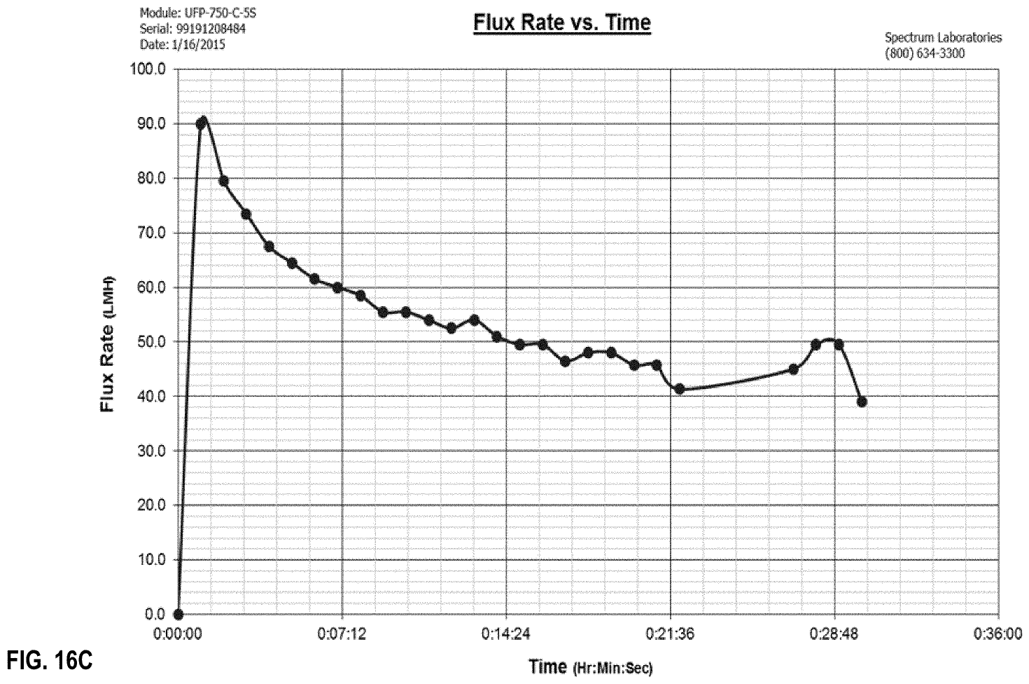

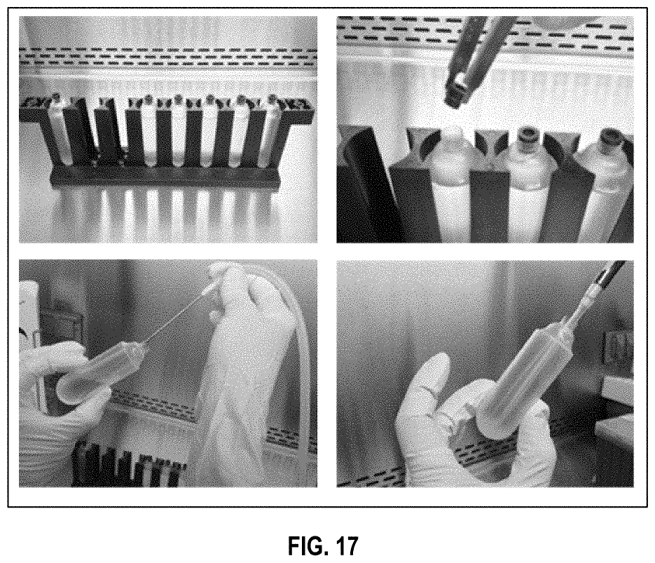

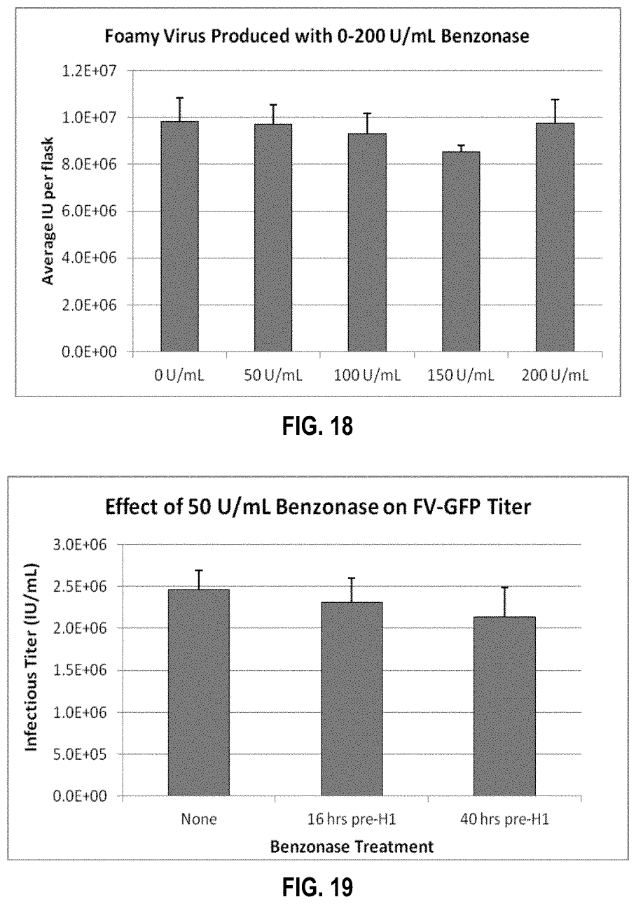

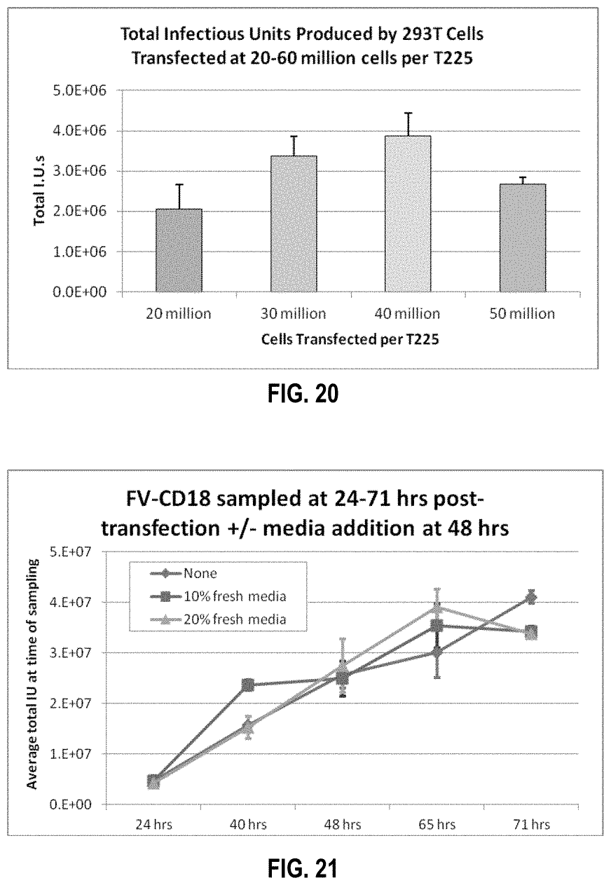

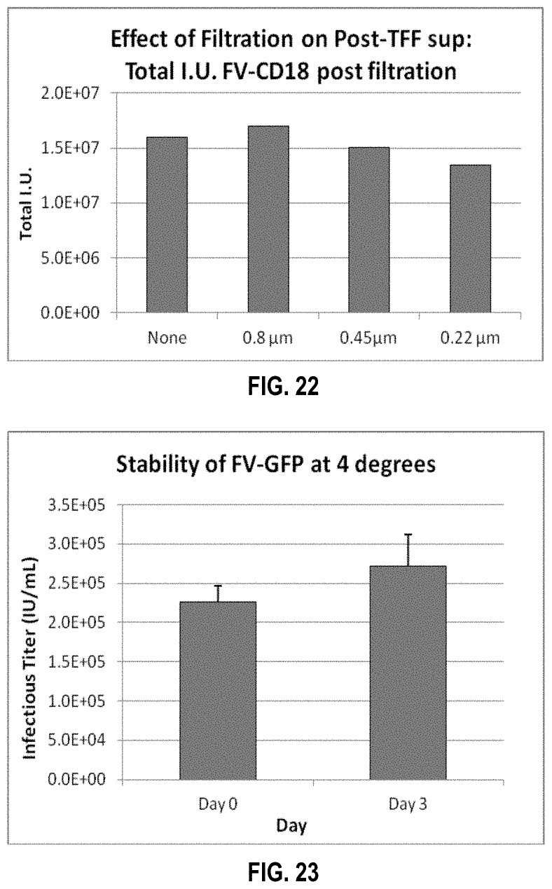

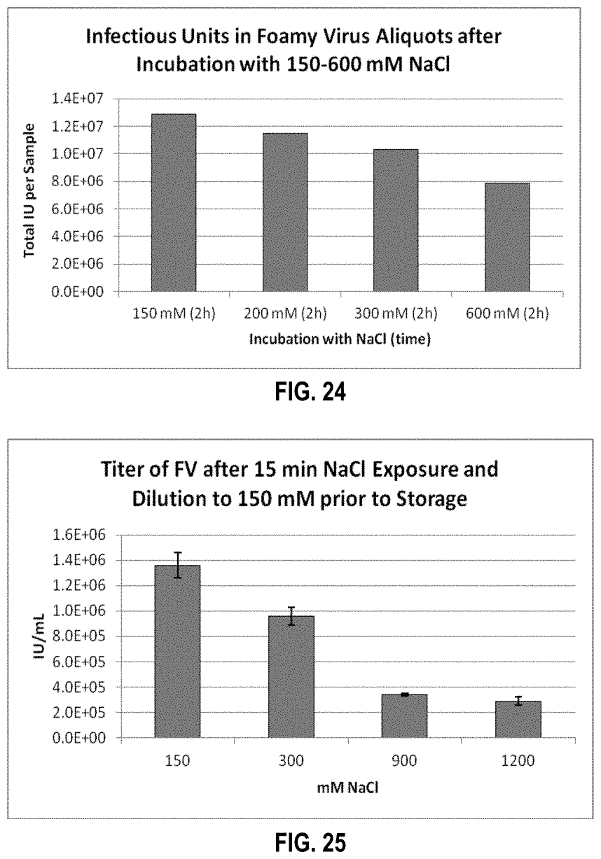

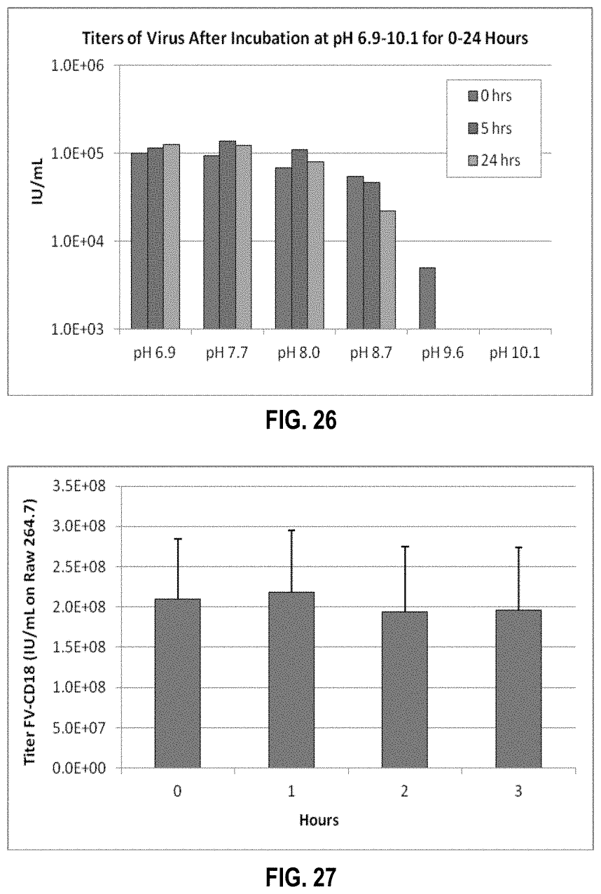

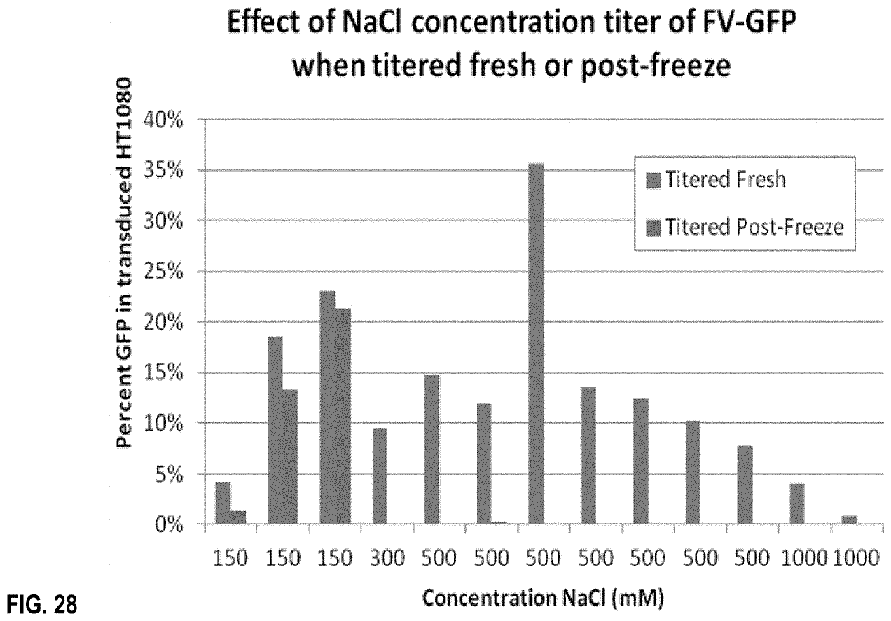

### DETAILED DESCRIPTION

Successful genetic correction of diseases, mediated by hematopoietic stem cells (HSCs) or alternate dividing cell types, depends upon stable integration of the targeted gene into the genome of the cell. Integration is required to assure that upon cell division all daughter cells carry the corrected or therapeutic gene. An example that requires an integrating vector is the genetic modification of cells in the blood and bone marrow. In addition, upon integration transferred genes need to be sufficiently expressed to provide therapeutic quantities of the required protein(s). Although significant advances in vector design over the years have improved the efficacy of gene therapy, certain key obstacles have emerged as barriers to successful clinical application. Among those obstacles, vector genotoxicity is among the most formidable, as evidenced by the occurrence of gene therapy related leukemia in a trial with patients suffering from X-linked severe combined immunodeficiency (SCID-X1). It was shown that the genotoxic events in this trial were the result of activation of an oncogene by the integrated viral vector^{8,9}. The gene therapy vector used in this trial was based on gamma-retrovirus. Since then, considerable research has shown that gamma-retroviral vectors can be modified to largely prevent oncogene activation upon integration¹⁰. Lentiviral vectors are integrating vectors that can be used as an alternative to gamma-retroviral vectors. Like gamma-retroviral vectors, lentiviral vectors stably integrate into the genome of the target cell. Although lentiviral vectors do not show the tendency to integrate in gene-promoter rich regions, they do show a predominant integration in regions with active genes. In contrast, recently emerged viral vectors based on the human Foamy Virus (FV) show excellent gene transfer into hematopoietic cells in a large animal model and an almost random integration profile which even further reduces the chance of genotoxicity⁷. This makes the FV vector system the safest integrating viral vector system available to date for clinical application.

Compared to other integrating viral vectors, foamy virus (FV) vectors have distinct advantages as a gene transfer tool, including their non-pathogenicity, the ability to carry larger transgene cassettes, and increased stability of virus particles due to DNA genome formation within the virions. Applicant has demonstrated proof of principle of its therapeutic utility with the correction of canine Leukocyte Adhesion Deficiency (LAD) using autologous CD34⁺ cells transduced with FV vector carrying the canine CD18 gene, demonstrating its long-term safety and efficacy. However, infectious titers of FV-human(h)CD18 were low and not suitable for manufacturing of clinical-grade product. Disclosed herein is a scalable production and purification process that is capable of a 60-fold higher FV-hCD18 titers from approximately 1.7×10⁴ to 1.0×10⁶ infectious units (IU)/mL. Process development improvements included use of polyethylenimine (PEI)-based transfection, use of a codon-optimized gag, heparin affinity chromatography, tangential flow filtration, and ultracentrifugation, which reproducibly resulted in 5000-fold concentrated and purified virus, an overall yield of 19±3%, and final titers of 1-2×10⁹ IU/mL. Highly concentrated vector allowed reduction of final DMSO concentration, thereby avoiding DMSO-induced toxicity to CD34⁺ cells while maintaining high transduction efficiencies. This process development results in clinically relevant, high titer FV which can be scaled up for clinical grade production.

Foamy viruses (FVs), also known as spumaretroviruses, derive their name from the vacuolating foamy-like cytoplasm of productively infected cells and multinucleated syncytia. They are endemic in a number of mammals, including cats, cows, and captive non-human primates, but not found in humans. Despite their highly cytopathic nature in cell culture, they are not associated with any detectable disease in infected hosts.^{17,18} The development of leukemia in X-linked severe combined immunodeficiency patients^{19, 20} and occurrence of myelodysplastic syndrome in chronic granulomatous disease patients²¹ caused by gamma-retrovirus vector mediated insertional mutagenesis after ex vivo stem cell gene therapy, has stimulated the development of vectors with improved safety profiles for clinical application. FVs have several distinct advantages over other integrating viral vectors such as gamma-retroviruses and lentiviruses as a gene transfer tool.^{22,23} These include a large packaging capacity (up to 12 Kb) and a broad host and cell-type tropism.^{17,18} Furthermore, FVs can efficiently transduce quiescent cells, since the FV genome can persist in a stable form as cDNA in growth-arrested cells/quiescent cells and can integrate into the host genome when the cells exit the GO phase of the cell cycle.²⁴ In addition, as compared to gamma-retrovirus or lentivirus, FV has a safer integration profile with lower risk of insertional mutagenesis.²⁵⁻²⁷ FV vectors have been used to correct genetic disorders of hematopoietic stem cells (HSCs) in several animal models, including leukocyte adhesion deficiency (LAD) in dogs,^{25, 28} and Wiskott-Aldrich syndrome (WAS), Fanconi anemia, and X-linked chronic granulomatous disease in mice.²⁹⁻³¹

Patients with LAD type 1 (LAD-1) and dogs with canine LAD (CLAD) suffer from recurrent and life-threatening bacterial infections.^{32,33} Both diseases are caused by mutations in the leukocyte integrin CD18 subunit that prevent the formation and surface expression of CD11/CD18 heterodimeric adhesion molecules resulting in an inability of leukocytes to adhere to the endothelium and migrate towards the sites of infection.³⁴

Successful gene therapy of CLAD was demonstrated in four dogs transplanted with autologous CD34+ cells transduced by FV vectors expressing canine CD18.^{25,28} However, the low titers typically obtained with FV vectors^{22,23}, have precluded their use for clinical application in LAD-1 patients. In addition, processes used previously were not scalable and not compatible with the large-scale manufacturing needed for clinical application. Major obstacles for scale-up of FV vector production and purification include: (i) the low titer of calcium phosphate-mediated transfection commonly used in gene transfer vector production^{22,23}, (ii) the limited stability of FV vectors in ambient or high temperature, acidic or basic pH, and high salt concentrations, (iii) their sensitivity to shear forces, and (iv) the necessity to freeze FV vectors in 5% DMSO and consequently to significantly dilute the vector to minimize toxicity to stem and progenitor cells during transduction.

Foamy Virus Vectors

Applicant has previously described methods for large scale manufacturing of gamma-retrovirus and lentivirus for clinical application in U.S. Patent Application 61/847,897 filed Jul. 18, 2013 (inventor Van Der Loo), published as WO 2015/010030 and as published¹⁰⁻¹². The methods described for gamma-retrovirus and lentivirus were used as a starting point for developing the manufacturing process for FV vector. One of the draw backs of the FV vector system is that FV vectors are stored at −80° C. in the presence of 5% Dimethyl sulfoxide (DMSO) to enhance stability and maximize recovery of infectious particles. DMSO, in turn, has shown to be toxic to HSCs at concentrations >0.1%. Consequently, one challenge was to design a manufacturing method capable of generating FV vector at a very high concentration (5000-fold); high enough to be able to dilute the material 50-fold at the point-of-use from 5% to 0.1% DMSO to prevent cell toxicity while maintaining transduction efficacy. Other challenges included the limited tolerance of the FV particle to high concentrations of salt (>150 mM) and high pH (>pH 8.0) which restricts the options available for purification and concentration. Here, Applicant discloses one or more methods for the large scale manufacturing of FV vector for clinical application. Applicant has found that the process of manufacturing high titer vector can be accomplished via alterations of one or more steps in the process including cell culture, the plasmids, transfection method, the harvest strategy, purification approach, and/or concentration methods.

In one aspect, a method of preparing FV vector particles is disclosed. The method may comprise the steps of:

a. transfecting a population of eukaryotic cells by contacting the population of eukaryotic cells with one or more transfection reagents to form a transfection mixture, and incubating the transfection mixture to form a transfected cell population;

b. harvesting the FV vector particles from the transfected cell population, wherein the harvesting step may be carried out about 70 hours to about 100 hours, or about 70 hours to about 90 hours, or about 70 hours to about 80 hours, or about 72 hours to about 75 hours, post-transfection;

c. purifying the FV vector particles;

d. concentrating the FV vector particles.

Pre-Seeding

In one aspect, the method may further comprise a pre-seeding step, wherein the population of eukaryotic cells may be pre-seeded for about 20 to about 30 hours, or about 24 hours, prior to the transfecting step.

In one aspect, the pre-seeding may be carried out until the population of eukaryotic cells achieves a cell density of from about 1×10⁵ cells/cm2 to about 2×10⁵ cells/cm² or about 1.8×10⁵ cells/cm².

In one aspect, the pre-seeding step may comprise the step of plating eukaryotic cells 1 day prior to PEI transfection with fresh media.

In one aspect, the pre-seeding step may comprise adding poly-L-lysine in an amount sufficient to pre-coat tissue culture plastic with about 3.5 to about 10 mL per 225 cm² surface area, preferably at a concentration of about 0.01%.

Transfection

In one aspect, the one or more transfection reagents may comprise vector plasmid and a plasmid comprising codon optimized pCiGAGopt. Codon optimization is readily understood by one of ordinary skill in the art, and may be performed by a third party, for example, as described at https://www.idtdna.com/CodonOpt.

In one aspect, the transfecting step may be carried out in the presence of about 10% (vol/vol) fetal bovine serum and about 0.4% (vol/vol) PEIPro.

In one aspect, the transfecting step may be carried out in the presence of about 10% fetal bovine serum, calcium phosphate, butyrate, and chloroquine.

In one aspect, the transfection mixture may be incubated for about 10 to about 20 minutes at ambient temperature (20-24° C.), preferably for about 10 minutes.

In one aspect, the transfection mixture may be maintained in the initial media until the day of harvest.

In one aspect, the pH of the transfection mixture may be less than about 8.

Purifying/Concentrating

In one aspect, the FV vector particles may be subjected to a filtration step.

In one aspect, the transfected cell population may be contacted with benzonase at a concentration of from about 50 to about 200 U/mL, preferably about 50 U/mL in the presence of about 10 mM MgCl₂ for a period of from about 2 to about 6 hours, preferably about 4 hours prior to a filtration step in one instance, and for 16 to 40 hours prior to vector harvest in another.

In one aspect, the FV vector particles may be isolated using a heparin column.

In one aspect, the method further may comprise the step of concentrating the FV vector particles using tangential flow filtration.

In one aspect, the method may further comprise the step of concentrating the FV vector particles, followed by dilution to about 140 mM to about 160 mM, preferably about 150 mM NaCl.

In one aspect, the method may further comprise the step of concentrating the FV vector particles using tangential flow filtration.

In one aspect, the method may further comprise the step of concentrating the FV vector particles using ultracentrifugation.

Storage

In one aspect, the FV vector particles may be stored at a temperature of about −70° C. to about −90° C., preferably about −80° C. in the presence of DMSO.

In one aspect, the FV vector particles may be stored frozen in the presence of from about 3 to about 5% DMSO, preferably about 5% DMSO.

In one aspect, a method of obtaining an increased titer of FV vector particles is disclosed. In this aspect, the method may comprise the steps of:

a. pre-seeding a population of eukaryotic cells for about 20 to about 30 hours, or about 24 hours, wherein the pre-seeding may be carried out until the population of eukaryotic cells achieves a cell density of from about 1×10⁵ cells/cm² to about 2×10⁵ cells/cm² or about 1.8×10⁵ cells/cm²;

b. transfecting a population of eukaryotic cells by contacting the population of eukaryotic cells with one or more transfection reagents, wherein the one or more transfection reagents may comprise a vector and a plasmid comprising codon optimized pCiGAGopt, wherein the plasmid may be used at a concentration of about 0.16 to about 10.4 microgram per 75 cm² culture surface equivalent, preferably 0.65 microgram per 75 cm2 culture surface equivalent to form a transfection mixture, and incubating the transfection mixture to form a transfected cell population;

c. harvesting the FV vector particles from the transfected cell population, wherein the harvesting step may be carried out about 70 hours to about 100 hours, or about 70 hours to about 90 hours, or about 70 hours to about 80 hours, or about 72 hours to about 75 hours, post-transfection;

d. purifying the FV vector particles, wherein the purification step may comprise use of a media comprising heparin;

e. concentrating the FV vector particles;

f. diluting the FV vector particles to about 150 mM. NaCl.

### Examples

Cells and Media

High-titer FV vector was generated by transfection of 293T cells using Polyethylenimine (PEI), with a mixture of vector and packaging plasmids in DMEM media in the presence of 10% Fetal Bovine Serum (FBS). Transfections were done in tissue culture plastic or in a bioreactor, as previously described for gamma-retrovirus and lentivirus (U.S. Patent Application 61/847,897, WO 2015/010030). Briefly, 293T cells are grown in DMEM with 10% FBS at 37° C., 5% CO₂ in a humidified incubator, harvested and seeded the day prior to transfection at 8×10⁴ cells/cm² in tissue culture plastic treated for 10 minutes at ambient temperature with Poly-L-Lysine Hydrobromide (PLL; Sigma-Aldrich, Mol Wt 150-300 kDa). Cells were maintained at 37° C., 5% CO₂ in a humidified incubator until transfection the next day. FBS concentrations ranging between 2-10% were less optimal as compared to 10% yielding titers several-fold lower. Also, the use of serum-free media, with or without supplementation with lipid (Gibco, Chemically-Defined Lipid), yielded titers lower compared to media with 10% FBS. Similarly, cultures initiated in tissue culture plastic without PLL yielded a lower titer. The length of incubation with PLL could be varied from 10 minutes to overnight without impact on viral titer. Seeding the day prior to transfection resulted in higher titers than seeding 3-days prior to transfection. Comparison of a range of cell densities from 20 to 60 million cells per T225 flask showed 40 million cells per T225 tissue culture flask (or 1.8×10⁵ cells/cm²) at the time of transfection to be optimal for FV.

Plasmids

Plasmids used for transfection included the FV vector plasmid (pΔΦ.SF.GFPpre or pΔΦ.MSCV.huCD18) and three packaging plasmids (pCIGSΔΨ or pCiGAGopt, pCIPS and pCIES). Plasmids were provided by Dr. D. Russell, from the University of Washington in Seattle. Plasmids pCIGSΔΨ, pCIPS and pCIES are described by Russell (13, 14). Plasmid pCiGAGopt is the codon-optimized form of pCIGSΔΨ. Applicant has found that the codon-optimized plasmid pCiGAGopt generated significantly higher titer as compared to the non-optimized pCIGSΔΨ. However, when at the same concentration, the codon-optimized plasmid pCiGAGopt appeared toxic generating a lower titer (not shown). Testing of a range of concentrations from 10.4 microgram to 0.163 microgram of pCiGAGopt per T75 flask showed 0.65 microgram (a 16-fold dilution from the amount used for the non-optimized pCIGSΔΨ) to be optimal, yielding titers up to 4 times higher as compared to the use of 10.4 microgram of pCIGSΔΨ. For the optimal and claimed optimal manufacturing method, all transfections were done using pCiGAGopt. Any codon-optimized Gag expression construct may be used for the instant methods. The use of one or more codon-optimized helper plasmids, including the Gag expression construct, Pol expression construct, and Env expression construct, enhances foamy viral vector titer as compared to non-codon optimized expression constructs.

Transfection

Cells seeded the day before were transfected with vector and packaging plasmids using PEIPro, a proprietary formulation of polyethylenimine (PEI) from Polyplus-transfection SA (New York, N.Y.). Vector plasmid and packaging plasmids pCiGAGopt, pCIPS and pCIES were mixed at a ratio of 15:1:2:1. For transfection of four S-layer Corning CellSTACKS (available from Sigma Aldrich, http://www.sigmaaldrich.com/labware/labware-products.html?TablePage=17192211), a total of 2.2 mg plasmid was added to plain DMEM in a volume of 42.2 mL. Similarly, 11.9 mL of PEI was mixed with plain DMEM in a final volume of 42.2 mL. The plasmid and PEI mixtures were then combined to a total volume of 84.4 mL. The amount of PEI to add had been tested using a range from 25 microliter to 80 microliter per T75 tissue culture flask equivalent. PEI at 70 microliter per T75 tissue culture flask equivalent was found to be optimal yielding approximately 3 to 4-fold more infectious FV as compared to 25 microliter. Cells transfected with the optimized amount of PEI yielded titers that were 4 to 5-fold higher as compared to transfection with calcium phosphate using a protocol optimized for gamma-retrovirus and lentivirus as previously described (U.S. Patent Application 61/847,897). In a separate set of experiments, the presence of 25 μM of chloroquine in the transfection mix, and induction with 10 mM of Butyrate at 16-19 hours post-transfection, were found to be optimal for FV titer when using calcium phosphate (not shown). However, considering that PEI transfection yielded higher titer, in this aspect, the protocol did not include calcium phosphate, chloroquine or butyrate. The 84.4 mL transfection mixture was incubated for 10-20 minutes at ambient temperature and divided over four 1 Liter bottles. When comparing incubation times ranging from 10 to 20 minutes, 10 minutes was found to be optimal yielding twice the amount of FV as compared to 20 minutes. To each 1 Liter bottle, DMEM with 10% FBS was added to a total volume of 750 mL. The final concentration of reagents at the time of infection was approximately 0.7 microgram of total plasmid per mL of culture media and 0.4% (vol/vol) PEIPro. To reduce concentration of phenol red in subsequent purification steps, DMEM used for transfection was phenol-red free. CellSTACKS seeded the day prior were removed from the incubator. From each CellSTACK, the existing growth media was removed and 750 mL of media and transfection mixture from the bottle added to the CellSTACK. Cell STACKS were placed back in the incubator for continued incubation at 37° C., 5% CO₂ in a humidified incubator.

Media Change

As previously described by Applicant with respect to the protocol for gamma-retrovirus and lentivirus production, (U.S. Patent Application 61/847,897), post-transfection media change was used to limit toxicity from the transfection process using calcium phosphate and to reduce the amount of residual plasmid in the product. PEI, on the other hand, does not cause toxicity and a media change is not required. A comparison of cultures transfected with PEI with or without media change the next day at 19 hours post-transfection, showed that a media change was actually detrimental to FV titer and dramatically reduced titer by approximately 5-fold. This was unexpected and opposite to what was observed with gamma-retrovirus and lentivirus (U.S. Patent Application 61/847,897). Therefore, post-transfection, cells were maintained without further manipulation until the day of harvest. Addition of 10% or 20% fresh media to the existing culture at 48 hours post-transfection did not enhance titer indicating that cells were not starved of essential factors present in the media prior to supernate harvest.

Harvest

Applicant found that, evaluating the amount of vector produced post-transfection at various time points, the highest amount of virus could be harvested at 74 to 93 hours post-transfection. Harvesting at two time points, at 40 hours for harvest 1 and 64 hours for harvest 2, revealed that harvest 2 was very low as compared to harvest one. The cumulative amount of vector collected at 40 and 64 hours post-transfection was lower compared to the amount harvested in a single harvest at a later time point. Based on the above, a single harvest at approximately 3 days post-transfection was found to be optimal.

Benzonase

Benzonase may be used to reduce the amount of intact residual plasmid in the final product. In the original protocol, modeled after what was described for gamma-retrovirus and lentivirus (U.S. Patent Application 61/847,897), transfected cells were treated with 50 U/mL Benzonase and 10 mM MgCl₂ prior to vector supernate harvest. A comparison of 16 versus 40 hour treatment showed that neither treatment negatively affected FV titer. Similarly, incubation with Benzonase for 16 hours at concentrations ranging from 0 to 200 U/mL did also not negatively affect FV titer. However, addition of Benzonase did require manipulation of the CellSTACKS to ensure Benzonase was mixed well and distributed equally among the multiple layers in the Cell STACK. Since the optimized manufacturing process caused cell layers to be fragile, presumably as a result of the high amount of virus produced, Applicant found it preferred to harvest vector first and then treat with Benzonase to not damage the cell layer prior to harvest. Cell supernate was treated with 50 U/mL Benzonase for 4 hours in the presence of 10 mM MgCl₂. A comparison of cell supernate treated with benzonase prior to or after 0.45 micron filtration showed that treatment prior to 0.45 micron filtration yielded a higher titer. Consequently, in one aspect, supernate may be harvested, treated with 50 U/mL Benzonase in the presence of 10 mM MgCl₂, and then filtered through a 0.45 micron Leukocyte Reduction Filter (LRF; Pall Corporation). In another aspect, transfected cells are treated approximately 16 to 40 hours prior to harvest with 50 U/mL Benzonase in the presence of 10 mM MgCl₂.

FV Stability

Applicant found FV vector supernate found to be relatively stable with no observable loss of infectious titer over a period of three days when stored at 4° C. Storage, on the other hand, at room temperature showed a reduction in FV titer. Similarly, FV vector supernate concentrated by TFF held overnight at 4° C. showed 98% and 96% recovery of infectious titer. FV vector was found to be unstable in high concentrations of NaCl (>150 mM) and at high pH (>pH 8.0). Also, FV vector was not stable when stored at or below −70° C. unless in the presence of 5% DMSO. Lower concentrations of DMSO (3% and 1%) showed a reduced recovery of infectious virus. Notably and unexpectedly, −80° C. storage of FV samples in the presence of 300 mM NaCl or higher and 5% DMSO rendered FV completely and irreversibly non-infectious.

Heparin Column Chromatography

Based on the identification of the cell membrane-associated heparin sulfate receptor as the receptor for human foamy virus by Md Nasimuzzaman, (15), and the binding of FV particles to a POROS-Heparin column at a small scale by others (16), FV generated using the protocol as described above was subsequently captured on a POROS-Heparin chromatography column, available from life Technologies, a Thermo Fisher Scientific Brand, www.lifetechnologies.com. A ratio of 1 mL of POROS-Heparin resin effectively bound FV from 100 mL of clarified supernate with minimal breakthrough using a two minute residence time. The column was equilibrated with 5 column volumes (CV) of 150 mM NaCl, 20 mM Phosphate Buffer, loaded with clarified supernate, washed with 10 CVs of 150 mM NaCl, 20 mM Phosphate Buffer, and eluted with 10 CVs of 600 mM NaCl buffer, 20 mM Phosphate Buffer. Salt stability testing showed that FV infectivity was affected by increased salt concentration in a time-dependent manner. Therefore, the material captured from the first 3 column volumes post-elution was diluted immediately post-elution at a ratio of 1:4 using 20 mM Phosphate Buffer to achieve a physiological concentration of 150 mM NaCl prior to continuing.

Tangential Flow Filtration

Applicant has demonstrated that FV can be efficiently concentrated directly from cell supernate by Tangential Flow Filtration (TFF) using a Polysulfone membrane cartridge from GE Healthcare Life Sciences at http://www.gelifesciences.com with a 750 kDa nominal molecular weight cutoff and 0.5 or 1 mm inner diameter (i.d.). This strategy allows for concentration as the first step followed by chromatography column purification. However, comparison of media with 2, 5 and 10% of FBS showed that increased serum required a higher shear (3000-4000 s-1) during the TFF run to prevent membrane fouling. Higher shears reduced the recovery of infectious FV from 78±5% at a shear of 742±16 (Avg±SEM, n=16) to 60±8% at a shear of 3768±32 (Avg±SEM, n=6). Since irradiated FBS, stored frozen and thawed, is known to contain a small amount of denatured protein, Applicant hypothesized that these may contribute to membrane fouling. However, pre-filtration of media with 10% FBS used for transfection and harvest at 0.2 and 0.1 micron did not reduce membrane fouling. Membrane fouling was limited at a reduced FBS concentration, however, the use of 2% FBS reduces FV titer. To increase the recovery, eliminate clogging due to membrane fouling, and capture the highest amount of FV, the method was changed to start with chromatography capture of FV from clarified supernate, as described above, followed by TFF of the partially purified material. This strategy proved effective and allowed for the product to be concentrated efficiently by TFF post POROS-Heparin at lower shear showing minimal membrane fouling and high recovery. The process resulted in an average flux rate of 40-70 Liter/Square Meter/Hour (LMH) using a starting trans-membrane pressure (TMP) of 2 Pounds per Square Inch (PSI) and Shear of 2000-3000 s-1.

Ultracentrifugation

Finally, to achieve the highest possible concentration of FV, the post-TFF material was concentrated by ultracentrifuge (UC) at 19,000 RPM, 11° C., for 2 hours, using pre-sterilized Bell-Top tubes. Using long-range pipette tips, the pellet is resuspended into the media of choice, filtered at 0.2 micron (either after or prior to UC), and stored at −80° C. in the presence of 5% DMSO. Applicant found that FV purified by POROS-Heparin and TFF can be concentrated using ultracentrifugation with an average step recovery of 47±5% (Avg±SEM, n=7) to achieve a final concentration of FV vector of 5000-7000-fold as compared to unpurified cell supernate. The recovery of infectious FV for the entire process averaged 19% (range 3-26%).

Scope

The scope includes the experimental findings described that form the basis for the large scale manufacturing method of FV vector for research or for clinical application. This includes the large scale manufacturing of FV vectors in general as well as manufacturing of a FV vector for the treatment of human LAD. The examples listed include the use of two distinct FV vectors: one vector expressing the Enhanced Green Fluorescence Protein (EGFP), which allows for a rapid screening of infectious particles by testing transduced cells for the expression of EGFP, the other vector expressing the CD18 gene which is the vector which will ultimately be used for the treatment of human LAD. Although the method was developed using the two vectors described above, the findings are broadly applicable to the large scale and clinical manufacturing of all FV vectors. This includes the use of FV vectors for gene therapy application in both pre-clinical and clinical studies. The method developed is compatible with large scale manufacturing in compliance with current Good Manufacturing Practices (cGMP).

In this study, Applicant has successfully addressed each obstacle for large-scale manufacturing of FV vectors compatible with current good manufacturing practices (GMP). Applicant first improved vector production by optimizing transfection with the use of polyethylenimine (PEI) and by varying parameters of producer cell culture, plasmid concentration, and harvest time. Applicant next improved vector purification with the use of heparin affinity chromatography since heparan sulfate was identified as a receptor for FV^{36,36}, and chromatography-based purification methods are scalable and can be performed in a closed system compatible with production of clinical-grade vectors.^{37,38} Finally Applicant used tangential flow filtration (TFF) and ultra-centrifugation for the final step of vector concentration. This optimized process resulted in highly concentrated FV vectors carrying the human CD18 cDNA (FV-hCD18) that can now be scaled up for clinical application.

Optimization of Transfection Conditions to Maximize FV Titers.

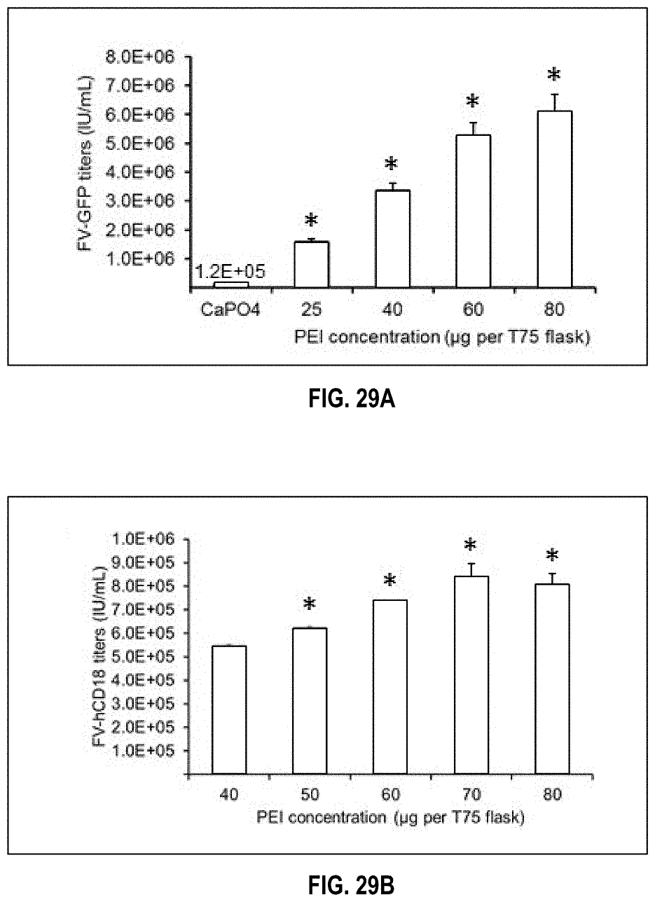

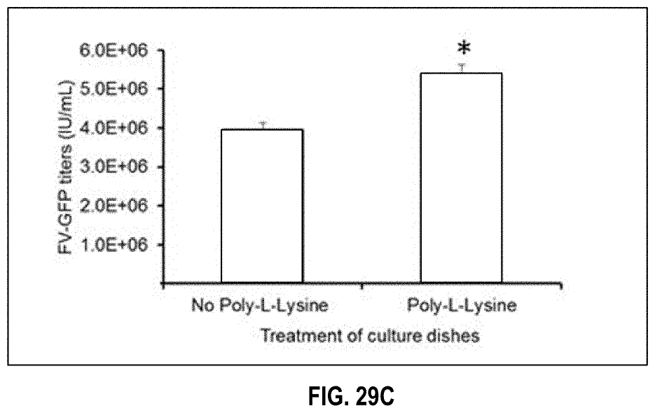

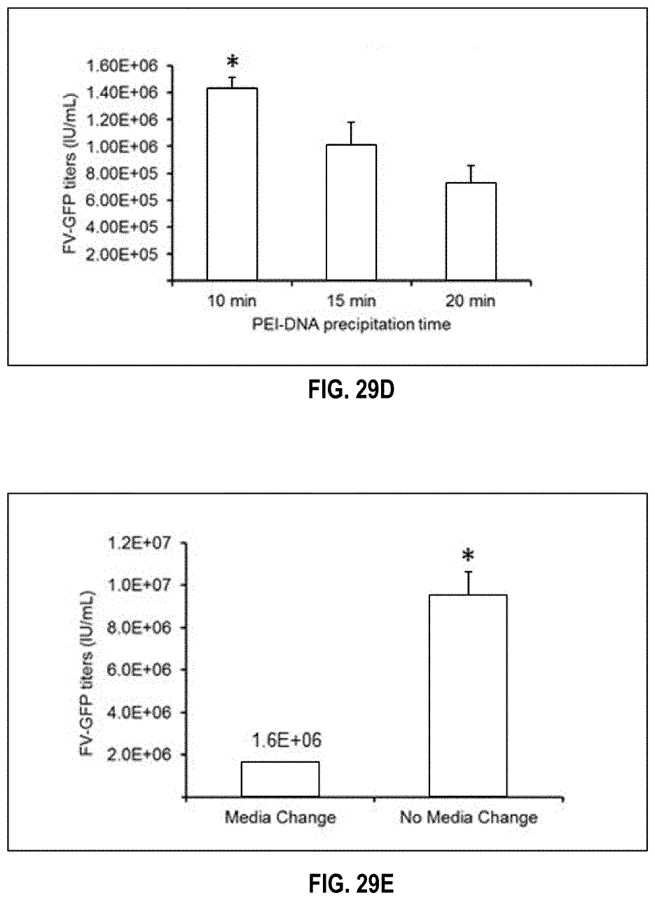

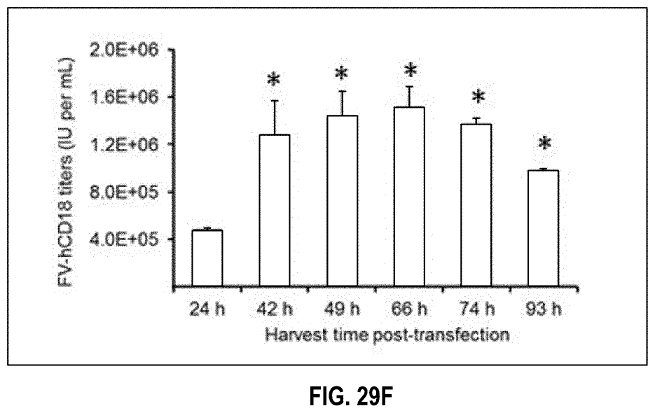

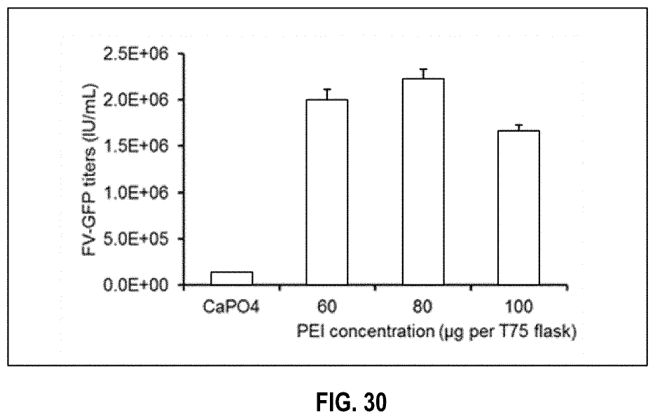

FV vectors were previously produced by calcium phosphate-mediated transient transfection of HEK239T cells with helper (gag, pol, and env) and gene transfer vector plasmids.²³ Unconcentrated titers of FV-GFP were 1.2±0.2×10⁵ IU/mL as determined on HT1080 cells and those of FV-hCD18 were 1.7±0.1×10⁴ IU/mL as determined on RAW264.7 cells. Applicant has recently published that PEI-mediated transfection resulted in up to a 50-fold increase in FV vector titers over calcium phosphate transfection.¹¹ In this study, PEI-mediated transfection was further optimized to maximize FV-GFP and FV-hCD18 vector titers. For both FV-GFP (FIG. 29A) and FV-hCD18 (FIG. 29B) vectors, titers improved with increasing concentrations of PEI, with a peak titer at 70-80 μg PEI per T75 flask. Further increases in PEI led to reduced titers (FIG. 30). After optimization, 70 μg of PEI per T75 flask was used during FV vector production in all experiments.

We also evaluated the effect of poly-L-lysine coating of culture plastic on FV-GFP vector production. Coating of culture plastic with 0.1% of poly-L-lysine prior to seeding HEK293T cells significantly increased FV vector titers (FIG. 29C). Although it has been suggested that a 15 min PEI-DNA precipitation time is optimal for high-titer FV vector production,²⁷ Applicant's current data showed that a 10 min precipitation time yielded the highest titers (FIG. 29D). Calcium phosphate-mediated transfection requires a medium change the next day to limit cellular toxicity and increase FV vector titers.^{30,40} Similarly, Applicant tested whether a change in medium after PEI-mediated transfection would also increase FV vector titers. Unexpectedly, this actually decreased FV vector titers by 2- to 5-fold (FIG. 29D). It is not clear whether this is due to a physiological response of the cells or related to a prolonged exposure to PEI and plasmid. Irrespectively, Applicant adopted a protocol in which the transfection medium containing PEI was not removed post-transfection but left with the cells until harvesting the vector. In addition, Applicant optimized the harvest time for FV vectors after transfection of the producer cells. FV-hCD18 vectors were sampled from 24 to 93 h post-transfection without medium change and titered. Harvesting of FV vectors around 66 h post-transfection yielded the highest titers (FIG. 29F).

Codon Optimized Gag Plasmid Further Increased FV Titers.

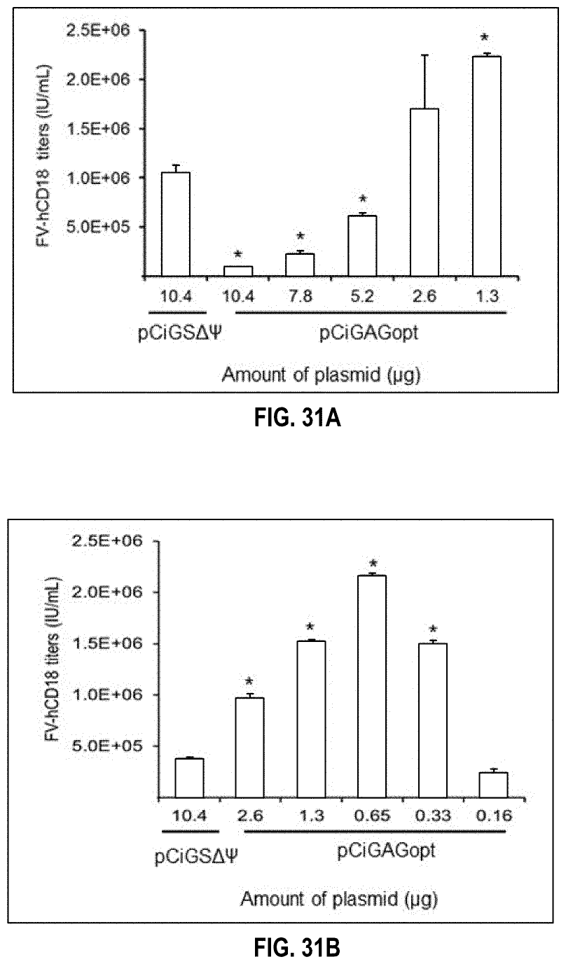

Applicant next compared pCiGSΔΨ (original gag) and pCiGAGopt (codon optimized gag) plasmids for FV-hCD18 vector production (FIG. 31A, FIG. 31B). Applicant previously observed that transfection of HEK293T cells with 10.4 μg of pCiGSΔΨ per T75 flask resulted in optimal FV-hCD18 vector titers (data not shown). However, significant toxicity to HEK293T was observed when the same amount of pCiGAGopt was transfected, resulting in a 10-fold reduction in FV-hCD18 titers (FIG. 31A). When amounts of pCiGAGopt were reduced from 10.4 to 1.3 μg per T75 flask in transfection, the titers of FV-hCD18 vectors increased proportionally (FIG. 31A). In a follow up study, the highest FV titer was obtained with 0.65 μg of pCiGAGopt plasmid (FIG. 31B). Thus, the use of codon optimized gag resulted in doubling of the FV-hCD18 vector titers while using 16-fold less plasmid as compared to the previously optimized amount of pCiGSΔΨ.

Benzonase Treatment of Cultures Post-Transfection to Reduce Residual Plasmid.

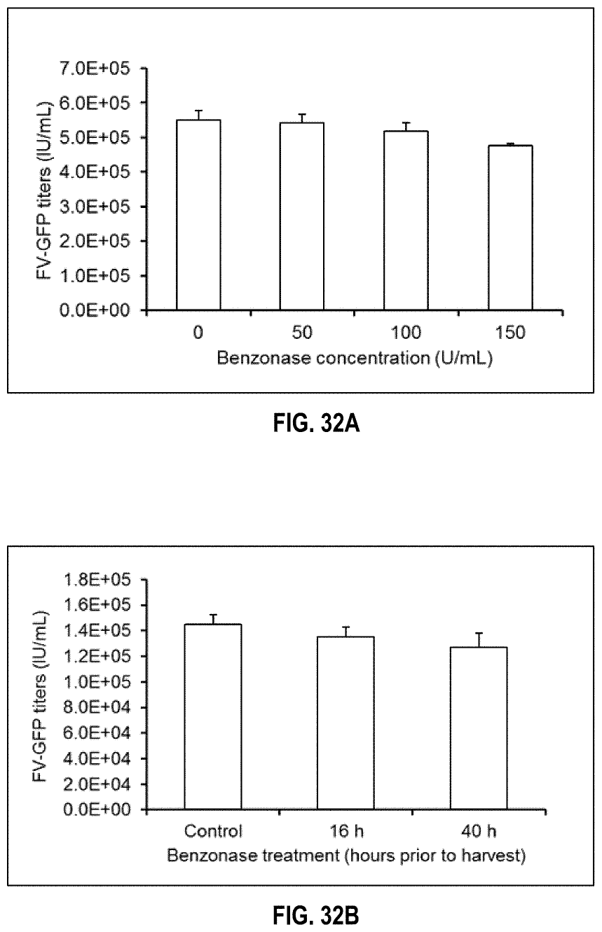

Benzonase endonuclease is commonly used to reduce the amount of residual plasmid and cellular genomic DNA and RNA in the vector product.⁴⁴ Treatment of FV vectors for 16 hours with increasing concentrations of Benzonase had only minimal impact on vector titers (FIG. 32A). Longer exposure (40 hours) with 50 U/mL Benzonase further reduced FV vector titers minimally (FIG. 32B). While differences were not statistically significant, Applicant chose a 16-hour exposure of Benzonase at 50 U/mL to limit the potential impact of Benzonase on FV titers. Overall, when all optimized conditions are combined, non-purified and unconcentrated FV-hCD18 titers of approximately 1×10⁶ IU/mL were consistently obtained, a 50-fold increase compared to titers obtained with the non-optimized protocol.

Purification of FV Vectors Using Heparin Affinity Chromatography.

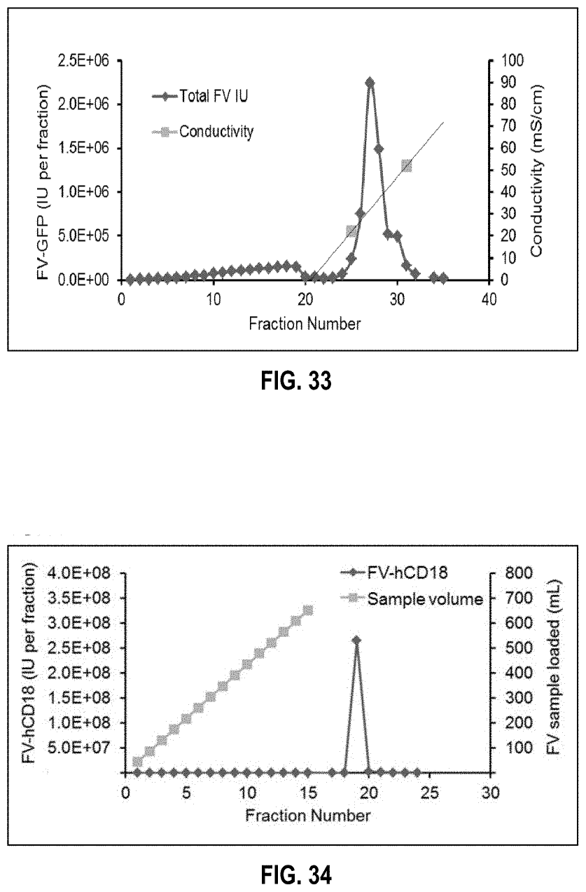

Since membrane-associated heparan sulfate, a heparin-related molecule, is a receptor for FV in cells,^{35,36} Applicant hypothesized that FV vector particles could be purified by heparin affinity chromatography. Applicant evaluated the binding, washing, and elution conditions needed for effective purification of FV vector. Prior to chromatography, nuclease-treated FV vector supernatants were filtered through a 0.45 μm filter to remove any coarse cellular debris. Vector supernatants were subsequently loaded onto a 7.9 mL bed volume POROS-OH 50 μm heparin affinity chromatography column at a linear flow rate of 267 cm/h and a residence time of 2.3 min. Faster flow rates and shorter residence time resulted in FV vector into the flow-through fraction (data not shown). After loading, the heparin column was washed with sodium phosphate or Tris-HCl buffer containing 150 mM sodium chloride (pH 7.0). The washing step was continued until the ultraviolet (UV) absorbance curve (280 nm) returned to baseline and became stabilized. To evaluate elution conditions, bound virus particles were eluted using a salt gradient from 100 mM to 1.0 M NaCl (pH 7.0). The optimal NaCl concentration for elution was determined based on the presence of infectious FV-GFP particles in individual chromatography fractions as measured on HT1080 cells and sample conductivity which correlated to NaCl concentration (FIG. 33). Applicant found that most of the FV-hCD18 was eluted at 600 mM of NaCl (FIG. 34). In addition, Applicant did not observe any significant loss of FV particles in the flow-through during loading and washing. The average recovery of FV vector in the elution fraction was 69±6% (n=5) as shown in Table 1.

TABLE 1

| | % of recovery | |
|---|---|---|
| Step | (Average ± SEM)* | n |
| Pre-load | 100 ± 0 | 5 |
| Loading | 4 ± 1.8 | 5 |
| Washing | 0 ± 0 | 5 |
| Elution | 69 ± 2.7 | 5 |

Concentration of FV vectors. Tangential flow filtration (TFF) is a rapid, efficient, and scalable method for concentration of small and large volumes of biological samples. Here, Applicant used TFF as a method to concentrate heparin affinity chromatography purified FV vector. Ultrafiltration was performed by recirculating the sample at 280 mL per min through a TFF cartridge with a 750 KDa nominal cut off using a trans-membrane pressure (TMP) between 5 and 6 psi. Vector particles were retained within the membrane whereas proteins smaller than 750 kDa were removed resulting in concentration and further purification of the vector. Vector was subsequently diafiltered using 100 mL of 150 mM NaCl, 25 mM Tris-HCl (pH 7.4) buffer. This step changed the concentration of salt to a physiological level. Using TFF, vectors were concentrated 20- to 30-fold with an average recovery of 89±13% (n=5) as shown in Table 2. The material was subsequently concentrated by ultracentrifugation at 50,000×g for 2 hours. Pellets were resuspended in final formulation buffer consisting of X-VIVO 10, 1% human serum albumin (HSA), and 5% DMSO. This last step concentrated the vector an additional 60-fold with 48±14% (n=5) recovery (Table 2). Overall, using the optimized conditions established for heparin affinity chromatography, TFF, and ultracentrifugation, the FV vectors were concentrated approximately 5,000-fold with a net recovery of 19±3.1% (n=5).

TABLE 2

| | Volume of | Processing | % of Step Recovery | |
|---|---|---|---|---|
| Step | Vector (mL) | Time | (Average ± SEM)* | n |
| Heparin column | 333.3 | 5 h | 69 ± 2.7 | 5 |
| TFF | 11.9 | 45 min | 89 ± 5.8 | 5 |
| 0.2 m filter | 11.9 | 15 min | 84 ± 4.5 | 5 |
| Ultracentrifugation | 0.2 | 2 h | 48 ± 6.3 | 5 |
| Net recovery | 0.2 | 8 h** | 19 ± 3.1 | 5 |

*Data represent mean and standard error of mean (SEM) of five independent experiments;
**Total Processing Time

FV-hCD18 Vector Transduction.

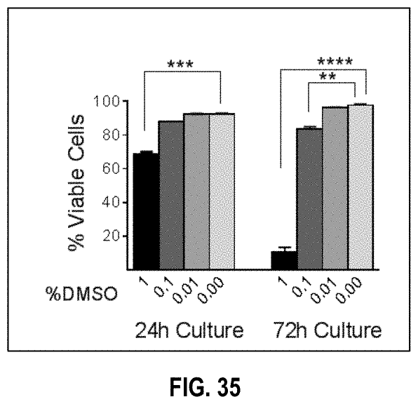

We tested the ability of purified FV-hCD18 vectors to transduce granulocyte-colony stimulating factor (G-CSF)-mobilized peripheral blood CD34+ cells obtained from two subjects diagnosed with LAD-1, using two independent FV-hCD18 vector pilot batches. CD34+ cells were cultured in the presence of cytokines on Retronectin-coated plates and transduced for 16 hours with concentrated and purified FV-hCD18 vector at various dilutions. Cells were washed and continued in culture for an additional 3 days to allow maximal detection of CD18 expression by flow cytometry. Since DMSO must be added for optimal recovery of FV vectors after cryopreservation, the highly concentrated FV-hCD18 vector was diluted to reduce DMSO concentration to ≤0.1% to limit the toxicity to CD34+ cells during transduction. Increasing doses of DMSO, especially with a prolonged exposure are well known to be toxic to murine and human hematopoietic cells and other types of stem cells, including human embryonic stem cells.⁴²⁻⁴⁴ Applicant confirmed these results and observed reduced viable CD34+ cells when DMSO concentrations exceeded 0.1% (FIG. 35). For both subjects, percentages of transduction in bulk CD34⁺ cells increased proportionally with increasing volumes of FV vector. Subject 1 has a moderate clinical phenotype and 18.7% of CD34+ cells expressed CD18 at baseline; CD18+ cells increased to 77.4% (i.e. 59% over baseline CD18 expression) after transduction at the highest MOI of FV vector tested. This level was similar to baseline CD18+ cells (87.3%) measured in MPB CD34+ cells from a healthy subject. In subject 2 with a severe phenotype, no CD18+ cells were detected at baseline. Up to 26.4% and 21.2% of LAD-1 CD34+ cells expressed CD18 after transduction with FV vector batch 1 and 2, respectively. For both subjects, FV-hCD18 vector had negligible impact on cell viability and cell growth, as measured 3 days after transduction, compared to untransduced LAD-1 CD34+ cells. Overall, these experiments provide proof of principle that clinical-grade high-titer FV vectors can be produced and purified for efficient transduction of LAD CD34+ cells with minimal DMSO-related toxicities. The data will be published in Nasimuzzaman et al. 2016, Molecular Therapy—Methods & Clinical Development.

### Discussion

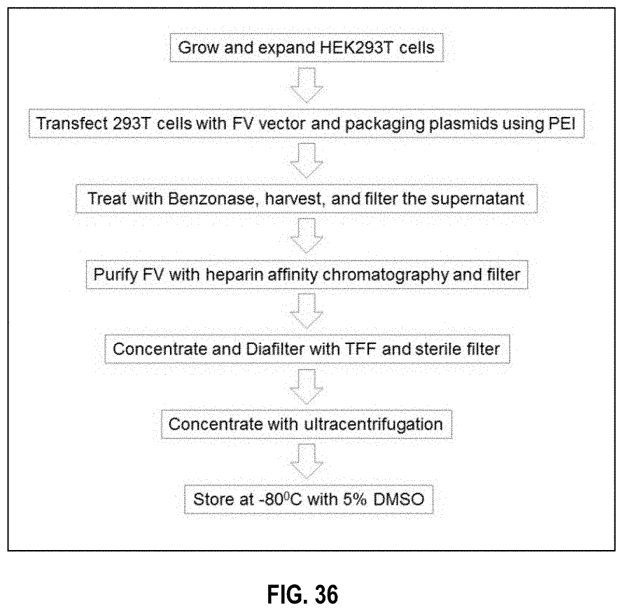

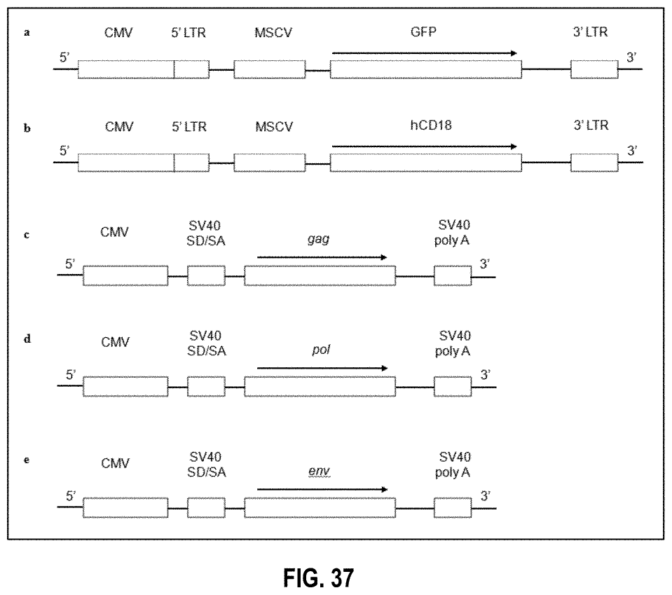

FV vectors represent a potentially safer alternative to currently used integrating viral vectors for gene therapy application. However, approaches customarily used to manufacture large-scale lentiviral vector for clinical application have resulted in low titers for FV vectors, 6, 7 hampering their clinical development. In this study, Applicant has presented process development with a step-by-step optimization of FV vector production and purification (FIG. 36).

PEI-mediated transfection of FV plasmids into HEK293T cells significantly increases the titers over those achieved with calcium phosphate.11 PEI has the ability to avoid trafficking to degradative lysosomes and its buffering capacity leads to osmotic swelling and rupture of endosomes, resulting in release of the vector particles into the cytoplasm and subsequently to the culture medium.⁴⁵ PEI has a high cationic charge density at physiological pH due to partial protonation of the amino groups in every third position. These amino groups form non-covalent complexes with negatively charged DNA, which leads to condensation and shielding of the negative charges, thereby allowing endocytosis into the cells, resulting in efficient transfection of vector producer cells.⁴⁶

Substantial plasmid DNA contamination is carried over in vector supernatants produced by transient transfection.⁴⁷ Plasmid DNA present in vector supernatants artificially increases the PCR-based titer of vectors and may be toxic to primary cells such as hematopoietic stem and progenitor cells (HSPCs) exposed to the concentrated vectors. Nucleic acids also result in increased supernatant viscosity which interferes with purification steps and reduces vector titers. Addition of Benzonase endonuclease during FV vector production allowed complete digestion of all forms of DNA and RNA to 5′-monophosphate terminated oligonucleotides 2 to 5 bases in length.⁴⁸ It is effective over a wide range of temperature and pH, and has no proteolytic activity, providing a simple approach to enhance FV vector production. Applicant's data supports that Benzonase endonuclease can be safely used in the manufacture of FV vector without significant loss of infectious titer.

Commonly used purification methods such as ultracentrifugation can precipitate FV particles along with cellular debris and serum proteins⁴⁹ which can be toxic to the target cells. Heparin affinity medium strongly binds only those particles that have affinity for heparin molecules.^{50,51} Unbound and loosely bound material present in FV supernatant, including cellular debris and serum proteins, elute in the flow-through during sample loading and washing with low salt containing buffer. The FV-heparin interaction is stable but reversible, requiring relatively low salt concentrations for dissociation as demonstrated here. This is important in considering the susceptibility of retroviruses to osmotic shock⁵¹ and limited stability of FV vectors in high salt (data not shown).

In contrast to conventional heparin affinity chromatography medium, POROS perfusion chromatography medium is engineered to have two discreet classes of pores. Large “through pores” allow convection flow to occur through the particles themselves, quickly carrying sample molecules to short “diffusive” pores inside. By reducing the distance over which diffusion needs to occur, the time required for sample molecules to interact with interior binding sites is also reduced. Diffusion is no longer limiting and flow rates can be dramatically increased without compromising resolution or capacity. Separation can be achieved at speeds up to 100-fold faster as compared to conventional heparin medium.⁵³ Applicant has carefully optimized the binding conditions and found POROS-Heparin to be superior in its ability to effectively capture FV particles as compared to Heparin-Sepharose medium such as Hi-Trap Heparin (data not shown).

The stability of vectors is strongly dependent on ultrafiltration parameters such as trans-membrane pressure, shear, and process run duration.⁵⁴ These parameters were optimized to maximize the concentration and recovery of FV vector. Although higher shear forces were helpful in reducing membrane fouling, these reduced vector titer (data not shown). Shear values between 2000 and 3000 s-1 resulted in an 89% recovery of infectious virus particles in Applicant's study. Membrane fouling was not an issue since most of the proteins were removed during the chromatography run. Since TFF is a closed system and ultracentrifugation tubes are sealed prior to the centrifugation step, both are compatible with clinical grade vector production.^{37.38}

After optimization of the process, two pilot batches of FV vectors produced showed 21-59% transduction efficiencies in G-CSF mobilized CD34+ cells derived from two LAD-1 subjects. In a preclinical gene therapy study of canine LAD, clinical benefit was observed with CD18 gene marking of 14-25% in bulk canine HSPCs after transduction,^{25,56} suggesting clinically relevant transduction efficiencies were achieved. FV vector cryopreservation necessitates 5% DMSO[1]⁵⁵ and, therefore, further escalation of FV vector volumes during transduction was not feasible due to DMSO-induced toxicity on target CD34+ cells (FIG. 35). Despite nearly identical VCN between subjects 1 and 2, expression of CD18 was quite different. The timing of flow cytometry for optimal CD18 gene expression in bulk CD34+ cells after transduction may vary between patients. For consistency, Applicant has chosen a period of 72 hours for both subjects but this may not be optimal for subject 2. Given the scarcity of LAD CD34+ cells, kinetic expression studies are impractical. Other explanations related to molecular differences (different mutations), phenotypic differences (subject 1: moderate; subject 2: severe), age differences (subject 1:19YO; subject 2:33 YO), or technical differences (widely different duration of cryopreservation of CD34+ cells, 4 years vs 1 month) between subjects 1 and 2 cannot be entirely ruled out. Given that transduction differed between the two patients tested here, it may be helpful to examine transduction efficiencies of patients CD34+ cells prior to gene therapy to optimize clinical transduction and even attempt correlating with heparan sulfate expression. If differences in transduction correlate with heparan sulfate levels, heparan sulfate expression may be used as a marker to predict transducibility. Based on the average FV titers using this methodology and the data in FIG. 4, where 450,000 cells transduced at 21.2% with 6 μL FV vector, transduction of 250 million cells (to treat a 50 kg individual with 5×10⁶ transduced cells/kg) will require approximately 3 mL of 5000-fold concentrated vector. This represents the equivalent of approximately 15 Liter of initial culture volume per patient, which is feasible from the manufacturing standpoint. In addition, canine data and some unpublished results by Applicant show that transduction efficiencies of approximately 20% are sufficient for long-term correction of LAD. Therefore, the FV vector production process described in this study paves the way to scale-up FV production for clinical manufacturing of FV-hCD18 vectors for a clinical trial in LAD-1 patients.

Materials and Methods

Plasmids.

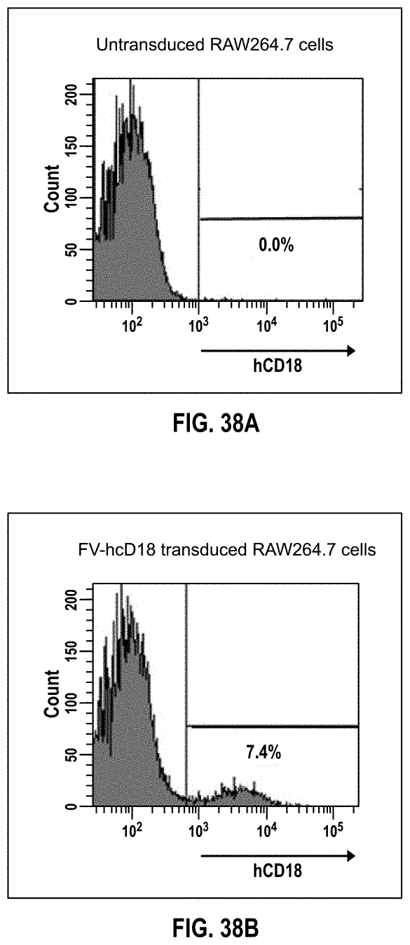

Self-inactivating FV gene-transfer vector plasmids pΔϕ-MSCV-green fluorescent protein (GFP) and pΔϕ-MSCV-huCD18, as well as packaging gene plasmids pCiGSΔΨ (gag), pCiGAGopt (codon optimized gag), pCiPS (pol), and pCiES (env) (FIGS. 38A and 38B) were constructed by Dr. David Russell.⁷ FV gene transfer, gag, pol, and env vector plasmids were used at a ratio of 14:14:2:1. When gag plasmid pCiGAGopt was used instead of pCiGSΔΨ, a 16-fold lower concentration of the plasmid was used for optimal titer. Plasmids were manufactured by Puresyn (Malvern, Pa.).

Cell Culture.

Human embryonic kidney cell line HEK293T, mouse macrophage cell line RAW 264.7, and human fibrosarcoma cell line HT1080 were grown in Dulbecco's modified Eagle's medium, high glucose, (DMEM; Invitrogen, San Diego, Calif.) supplemented with 10% fetal bovine serum (FBS), 1 mM L-glutamax, 1 mM sodium pyruvate, and 1 mM non-essential amino acids (Invitrogen, San Diego, Calif.). Human CD34⁺ cells from two LAD-1 patients were cultured in StemSpan Serum-Free Expansion Media (SFEM) II (STEMCELL Technologies, Vancouver, BC, Canada) containing penicillin-streptomycin and cytokines (hereafter referred to as CD34⁺ cell culture medium), including 300 ng/mL human stem cell factor, 100 ng/mL human thrombopoietin and 300 ng/mL human FLT3 ligand (all from PeproTech, Rocky Hill, N.J.). All cultures were performed at 37° C. and 5% CO₂ in a humidified incubator.

Vector Production.

In some experiments, FV vectors were produced by calcium phosphate-mediated transient transfection, as described previously.^{22,23,57} In most experiments, FV vectors were produced by polyethylenimine (PEI) (Polyplus-Transfection, France)-mediated transient transfection. HEK293T cells were seeded in growth media in tissue culture treated flasks or CellSTACKS pre-coated with poly-L-lysine (Sigma-Aldrich) at 0.1% (g/L) for 10 minutes at ambient temperature. For transfection, FV plasmids and PEI solution were diluted each in serum-free DMEM, combined and mixed by swirling. The mixture was incubated for 10-20 minutes (with 10 minutes being optimal) at ambient temperature to allow for the formation of a DNA-PEI precipitate. The used medium was removed from the cells and fresh growth medium containing the transfection reaction mixture was added. Transfected cells were cultured for approximately 48 hours and subsequently treated with 50 Units/mL of Benzonase endonuclease (Millipore, Billerica, Mass.) in media containing 10 mM MgCl2 at 37° C. for approximately 16 hours to digest residual plasmid, genomic DNA, and RNA. FV supernatants were harvested and clarified by passing through a leukocyte reduction filter (LRF; Pall) and 0.45 μm Gamma Gold filter (Millipore, Billerica, Mass.). FV supernatants were stored at −80° C. with 5% DMSO or purified immediately.

Vector Purification and Concentration.

Since FV reversibly binds heparin molecules, heparin affinity chromatography was used for the capture of FV vectors from media derived from transfected cultures. The resin, POROS Heparin (Applied Biosystems, San Diego, Calif.) contains an immobilized heparin functional group designed for high throughput purification of proteins or viruses with specific affinity for heparin. Filtered FV vector supernatants were loaded onto a POROS-OH 50 μm heparin column using an AKTAvant 150 (GE Healthcare, Piscataway, N.J.) chromatography system running with Unicorn 6.2 software, with a linear flow rate of 267 cm/h and residence time of 2.3 min. After loading, the column was washed with 20 mM sodium phosphate (pH 7.4) or 25 mM Tris-HCl (pH 7.4) buffer containing 150 mM sodium chloride. Washing continued until the ultraviolet (UV) absorbance curve (280 nm) returned to baseline and stabilized. Bound FV vector particles were eluted from the heparin column in 25 mM Tris-HCl containing 600 mM NaCl. Upon collection, vector was immediately diluted to a final concentration 150 mM NaCl using 25 mM Tris-HCl (pH 7.4). Post-chromatography, vector was clarified using a 0.45 micron filter and concentrated 25 to 30-fold using a 750 kDa tangential flow filtration (TFF) column (UFP-750-C-3MA, GE Healthcare, Piscataway, N.J.). Vector was diafiltered with a 10-fold excess of 25 mM Tris-HCl, pH 7.4, 150 mM NaCl. The concentrated retentate was sterile filtered through 0.22 μm pore size filter and subjected to ultracentrifugation in pre-sterilized ultra-centrifuge tubes (Beckman Coulter, Indianapolis, Ind.) at 50,000×g for 2 h at 11° C. using aseptic technique. The pellet containing the vector was resuspended by pipetting up and down in X-VIVO 10 (Lonza, Allendale, N.J.) containing 1% human serum albumin (HSA). The vector was resuspended in 5% DMSO (Sigma, St. Louis, Mo.) to obtain a concentration factor of approximately 5,000-fold as compared to the starting material. Vector was frozen on dry ice, and stored at −80° C.

Vector Titration.

Infectious titers of FV-GFP were determined using human HT1080 cells. Infectious titers of FV vector expressing CD18 cDNA were determined on RAW264.7 murine monocytic cell line. RAW264.7 cells express mouse but not human CD11/CD18. When transduced with FV vector expressing human CD18 cDNA, the human CD18 cross-heterodimerizes with mouse CD11. Transduced cells expressing the mouse/human hybrid CD11/CD18 are identified by flow cytometry using a fluorescently labeled mouse anti-human CD18 monoclonal antibody (FIG. 39). Since titers vary with the cell type, FV-GFP infectious titers were compared on both HT1080 and RAW cells, and found to be an order of magnitude higher in HT1080 cells, in general. Briefly, cells were seeded at 5×10⁴ cells/well in a 24-well plate one day before infection. FV vector supernatants were added to the cells at limiting dilution. Medium was replaced with fresh growth medium the next day. Three days post-transduction, cells were harvested and analyzed for GFP expression or stained with mouse anti-human CD18-APC (Clone 6.7, BD Biosciences, San Diego, Calif.) diluted in 1% bovine serum albumin (BSA) in PBS. Cells were washed with 1% BSA in PBS and analyzed on a flow cytometer (LSR-Fortessa, BD Biosciences). Titers (Infectious Units [IU] per ml) were calculated based on the number of cells at the time of infection, the dilution factor, and percentage of GFP+ or CD18+ cells.

Mobilization, Apheresis, and Purification of Human LAD-1 CD34+ Cells.

Two subjects with LAD-1 received G-CSF 10 μg/kg (Amgen, Thousand Oaks, Calif.) for 5 days, given as a single daily subcutaneous injection. Large volume (15 L) leukapheresis was initiated on the morning of day 5 of G-CSF administration, using a blood cell separator (Cobe Spectra, Terumo BCT, Lakewood, Colo.). The mononuclear cell (MNC) concentrates were enriched in CD34+ cells using a semi-automated CliniMACS Plus instrument (Miltenyi Biotec, Auburn, Calif.) and cryopreserved prior to transduction. All subjects gave written informed consent on treatment protocols approved by the Institutional Review Board (IRB) of the National Heart, Lung and Blood Institute (NHLBI), National Institutes of Health (NIH), in accordance with the Declaration of Helsinki.

Transduction of Human LAD-1 CD34+ Cells.

Human LAD-1 CD34+ cells (450,000 cells/well) were transduced with different volumes of FV-hCD18 in 300 μL CD34+ cell culture medium in 24-well tissue culture plates coated with RetroNectin 5 μg/cm² (TaKaRa, Shiga, Japan). Plates were subjected to spinoculation at 300×g for 5 min and incubated overnight (16-17 hrs) at 37° C. The following morning, FV vector supernatant was removed and fresh CD34+ cell culture medium was added. Three days post-transduction, cells were collected by gentle scraping, stained with anti-human CD18-FITC antibody (clone 6.7, BD Biosciences, San Jose, Calif.), and analyzed by flow cytometry using a LSR Fortessa instrument (BD Biosciences).

Real Time PCR Analysis for Vector Copy Number (VCN) Determination.

The presence of CD18 proviral sequences in genomic DNA isolated from CD34+ cells after transduction was determined using the ABI PRISM 7500 Real-Time PCR System (Life Technologies, Grand Island, N.Y.). Briefly, primers MSCV-F (5′-AGTCCTCCGATAGACTGC GT-3′), and CD18-R (5′-CTTCGTGCACTCCTGAGAGA-3′) amplified a vector-specific 123-bp fragment spanning the MSCV promoter and hCD18 cDNA. Amplification was detected with the MSCV-CD18 probe (5′-/56-FAM/TCTCCACCA/ZEN/TGCTGGGCCTG/3IABkFQ/-3′). The human albumin gene was used as an endogenous control for data normalization. Primers Hs Albumin-F (5′-GCT CTC CTG CCT GTT CTT TA-3′) and Hs Albumin R (5′-GGATTCTGTG CAGCATTTGG-3′) amplified a 204-bp fragment spanning the intron 11-exon 12 junction of the human albumin gene. Amplification was detected with the Hs Albumin probe (5′-/56-FAM/CCGTGGT CC/ZEN/TGAACCAGTTATGTGT/3IABkFQ/-3′). Amplification of plasmids containing cloned target sequences of MSCV-hCD18 or Hs Albumin intron 11-exon 12 junction was used to prepare a standard curve to quantify the number of FV-hCD18 vector integrations per diploid genome. For multiplex pPCR reactions, the FV- and albumin-specific amplicon primers were used in combination with the FAM-labeled, vector-specific TaqMan probe (MSCV-CD18) described above and the following albumin gene-specific TaqMan probe: 5′-/56-JOE NHS/CCGTGGTCC/ZEN/TGAACCAGTTATGTGT/3IABkFQ/-3′. Samples underwent denaturation at 95° C. for 10 minutes, followed by 40 cycles of amplification (15 seconds at 95° C., 1 minute at 60° C.).

Statistical analysis Statistical analysis was done using a two-tailed Student's t-test. A P value of ≤0.05 was considered statistically significant.

### REFERENCES

1 Kiem, H. P., Allen, J., Trobridge, G., Olson, E., Keyser, K., Peterson, L. and Russell, D. W. (2007) Foamy-virus-mediated gene transfer to canine repopulating cells. Blood, 109, 65-70.
2 Trobridge, G. D., Allen, J., Peterson, L., Ironside, C., Russell, D. W. and Kiem, H. P. (2009) Foamy and lentiviral vectors transduce canine long-term repopulating cells at similar efficiency. Hum Gene Ther, 20, 519-523.
3 Trobridge, G. D., Miller, D. G., Jacobs, M. A., Allen, J. M., Kiem, H. P., Kaul, R. and Russell, D. W. (2006) Foamy virus vector integration sites in normal human cells. Proc Natl Acad Sci USA, 103, 1498-1503.
4 Hendrie, P. C., Huo, Y., Stolitenko, R. B. and Russell, D. W. (2008) A rapid and quantitative assay for measuring neighboring gene activation by vector proviruses. Mol Ther, 16, 534-540.
5 Hirata, R. K., Miller, A. D., Andrews, R. G. and Russell, D. W. (1996) Transduction of hematopoietic cells by foamy virus vectors. Blood, 88, 3654-3661.
6 Bauer, T. R., Jr., Allen, J. M., Hai, M., Tuschong, L. M., Khan, I. F., Olson, E. M., Adler, R. L., Burkholder, T. H., Gu, Y. C., Russell, D. W. et al. (2008) Successful treatment of canine leukocyte adhesion deficiency by foamy virus vectors. Nat Med, 14, 93-97.
7 Bauer, T. R., Jr., Tuschong, L. M., Calvo, K. R., Shive, H. R., Burkholder, T. H., Karlsson, E. K., West, R. R., Russell, D. W. and Hickstein, D. D. (2013) Long-term follow-up of foamy viral vector-mediated gene therapy for canine leukocyte adhesion deficiency. Mol Ther, 21, 964-972.
8 Hacein-Bey-Abina, S., Von Kalle, C., Schmidt, M., McCormack, M. P., Wulffraat, N., Leboulch, P., Lim, A., Osborne, C. S., Pawliuk, R., Morillon, E. et al. (2003) LMO2-associated clonal T cell proliferation in two patients after gene therapy for SCID-X1. Science, 302, 415-419.

9 McCormack, M. P. and Rabbitts, T. H. (2004) Activation of the T-cell oncogene LMO2 after gene therapy for X-linked severe combined immunodeficiency. N Engl J Med, 350, 913-922.
10 Hacein-Bey-Abina, S., Pai, S. Y., Gaspar, H. B., Armant, M., Berry, C. C., Blanche, S., Bleesing, J., Blondeau, J., de Boer, H., Buckland, K. F. et al. (2014) A modified gamma-retrovirus vector for X-linked severe combined immunodeficiency. N Engl J Med, 371, 1407-1417.
11 van der Loo, J. C., Swaney, W. P., Grassman, E., Terwilliger, A., Higashimoto, T., Schambach, A., Baum, C., Thrasher, A. J., Williams, D. A., Nordling, D. L. et al. (2012) Scale-up and manufacturing of clinical-grade self-inactivating gamma-retroviral vectors by transient transfection. Gene Ther, 19, 246-254.
12 van der Loo, J. C., Swaney, W. P., Grassman, E., Terwilliger, A., Higashimoto, T., Schambach, A., Hacein-Bey-Abina, S., Nordling, D. L., Cavazzana-Calvo, M., Thrasher, A. J. et al. (2012) Critical variables affecting clinical-grade production of the self-inactivating gamma-retroviral vector for the treatment of X-linked severe combined immunodeficiency. Gene Ther, 19, 872-876.
13 Trobridge, G., Josephson, N., Vassilopoulos, G., Mac, J. and Russell, D. W. (2002) Improved foamy virus vectors with minimal viral sequences. Mol Ther, 6, 321-328.
14 Josephson, N.C., Trobridge, G. and Russell, D. W. (2004) Transduction of long-term and mobilized peripheral blood-derived NOD/SCID repopulating cells by foamy virus vectors. Hum Gene Ther, 15, 87-92.
15 Nasimuzzaman, M. and Persons, D. A. (2012) Cell Membrane-associated heparan sulfate is a receptor for prototype foamy virus in human, monkey, and rodent cells. Mol Ther, 20, 1158-1166.
16 Plochmann, K., Horn, A., Gschmack, E., Armbruster, N., Krieg, J., Wiktorowicz, T., Weber, C., Stirnnagel, K., Lindemann, D., Rethwilm, A. et al. (2012) Heparan sulfate is an attachment factor for foamy virus entry. J Virol, 86, 10028-10035.
17. Rethwilm, A (2007). Foamy virus vectors: an awaited alternative to gammaretro- and lentiviral vectors. Curr Gene Ther 7: 261-271.
18. Rethwilm, A (2010). Molecular biology of foamy viruses. Med Microbiol Immunol 199: 197-207.
19. Hacein-Bey-Abina, S, von Kalle, C, Schmidt, M, Le Deist, F, Wulffraat, N, McIntyre, E, et al. (2003). A serious adverse event after successful gene therapy for X-linked severe combined immunodeficiency. N Engl J Med 348: 255-256.
20. Hacein-Bey-Abina, S, Von Kalle, C, Schmidt, M, McCormack, M P, Wulffraat, N, Leboulch, P, et al. (2003). LMO2-associated clonal T cell proliferation in two patients after gene therapy for SCID-X1. Science 302: 415-419.
21. Stein, S, Ott, M G, Schultze-Strasser, S, Jauch, A, Burwinkel, B, Kinner, A, et al. (2010). Genomic instability and myelodysplasia with monosomy 7 consequent to EVI1 activation after gene therapy for chronic granulomatous disease. Nat Med 16: 198-204.
22. Trobridge, G D, and Russell, D W (1998). Helper-free foamy virus vectors. Human gene therapy 9: 2517-2525.
23. Trobridge, G, Josephson, N, Vassilopoulos, G, Mac, J, and Russell, D W (2002). Improved foamy virus vectors with minimal viral sequences. Molecular therapy: the journal of the American Society of Gene Therapy 6: 321-328.
24. Trobridge, G, and Russell, D W (2004). Cell cycle requirements for transduction by foamy virus vectors compared to those of oncovirus and lentivirus vectors. J Virol 78: 2327-2335.
25. Bauer, T R, Jr., Allen, J M, Hai, M, Tuschong, L M, Khan, I F, Olson, E M, et al. (2008). Successful treatment of canine leukocyte adhesion deficiency by foamy virus vectors. Nat Med 14: 93-97.
26. Trobridge, G D, Miller, D G, Jacobs, M A, Allen, J M, Kiem, H P, Kaul, R, et al. (2006). Foamy virus vector integration sites in normal human cells. Proc Natl Acad Sci USA 103: 1498-1503.
27. Nasimuzzaman, M, Kim, Y S, Wang, Y D, and Persons, D A (2014). High-titer foamy virus vector transduction and integration sites of human CD34(+) cell-derived SCID-repopulating cells. Molecular therapy Methods & clinical development 1: 14020.
28. Bauer, T R, Jr., Tuschong, L M, Calvo, K R, Shive, H R, Burkholder, T H, Karlsson, E K, et al. (2013). Long-term follow-up of foamy viral vector-mediated gene therapy for canine leukocyte adhesion deficiency. Molecular therapy: the journal of the American Society of Gene Therapy 21: 964-972.
29. Uchiyama, T, Adriani, M, Jagadeesh, G J, Paine, A, and Candotti, F (2012). Foamy virus vector-mediated gene correction of a mouse model of Wiskott-Aldrich syndrome. Molecular therapy: the journal of the American Society of Gene Therapy 20: 1270-1279.
30. Chatziandreou, I, Siapati, E K, and Vassilopoulos, G (2011). Genetic correction of X-linked chronic granulomatous disease with novel foamy virus vectors. Exp Hematol 39: 643-652.
31. Si, Y, Pulliam, A C, Linka, Y, Ciccone, S, Leurs, C, Yuan, J, et al. (2008). Overnight transduction with foamyviral vectors restores the long-term repopulating activity of Fancc−/− stem cells. Blood 112: 4458-4465.
32. Anderson, D C, and Springer, T A (1987). Leukocyte adhesion deficiency: an inherited defect in the Mac-1, LFA-1, and p 150, 95 glycoproteins. Annu Rev Med 38: 175-194.
33. Trowald-Wigh, G, Hakansson, L, Johannisson, A, Norrgren, L, and Hard of Segerstad, C (1992). Leucocyte adhesion protein deficiency in Irish setter dogs. Vet Immunol Immunopathol 32: 261-280.
34. Kijas, J M, Bauer, T R, Jr., Gafvert, S, Marklund, S, Trowald-Wigh, G, Johannisson, A, et al. (1999). A missense mutation in the beta-2 integrin gene (ITGB2) causes canine leukocyte adhesion deficiency. Genomics 61: 101-107.
35. Nasimuzzaman, M, and Persons, D A (2012). Cell Membrane-associated heparan sulfate is a receptor for prototype foamy virus in human, monkey, and rodent cells. Molecular therapy: the journal of the American Society of Gene Therapy 20: 1158-1166.
36. Plochmann, K, Horn, A, Gschmack, E, Armbruster, N, Krieg, J, Wiktorowicz, T, et al. (2012). Heparan sulfate is an attachment factor for foamy virus entry. J Virol 86: 10028-10035.
37. Burova, E, and Ioffe, E (2005). Chromatographic purification of recombinant adenoviral and adeno-associated viral vectors: methods and implications. Gene Ther 12 Suppl 1: S5-17.
38. Allay, J A, Sleep, S, Long, S, Tillman, D M, Clark, R, Carney, G, et al. (2011). Good manufacturing practice production of self-complementary serotype 8 adeno-associated viral vector for a hemophilia B clinical trial. Human gene therapy 22: 595-604.
39. Tiscornia, G, Singer, O, and Verma, I M (2006). Production and purification of lentiviral vectors. Nature protocols 1: 241-245.
40. Hanawa, H, Kelly, P F, Nathwani, A C, Persons, D A, Vandergriff, J A, Hargrove, P, et al. (2002). Comparison of various envelope proteins for their ability to pseudotype lentiviral vectors and transduce primitive hematopoietic cells from human blood. Molecular therapy: the journal of the American Society of Gene Therapy 5: 242-251.
41. Sastry, L, Xu, Y, Cooper, R, Pollok, K, and Cornetta, K (2004). Evaluation of plasmid DNA removal from lentiviral vectors by benzonase treatment. Human gene therapy 15: 221-226.
42. Rowley, S D, and Anderson, G L (1993). Effect of DMSO exposure without cryopreservation on hematopoietic progenitor cells. Bone Marrow Transplant 11: 389-393.
43. Han, D L, Liu, K Q, Guo, S S, Zhu, H L, Huang, C, and Wang, B H (2008). [Dose-effect relationship of DMSO and Tween 80 influencing the growth and viability of murine bone marrow-derived cells in vitro]. Zhongguo Shi Yan Xue Ye Xue Za Zhi 16: 377-380.
44. Pal, R, Mamidi, M K, Das, A K, and Bhonde, R (2012). Diverse effects of dimethyl sulfoxide (DMSO) on the differentiation potential of human embryonic stem cells. Arch Toxicol 86: 651-661.
45. Akinc, A, Thomas, M, Klibanov, A M, and Langer, R (2005). Exploring polyethylenimine-mediated DNA transfection and the proton sponge hypothesis. The journal of gene medicine 7: 657-663.
46. Boussif, O, Lezoualc'h, F, Zanta, M A, Mergny, M D, Scherman, D, Demeneix, B, et al. (1995). A versatile vector for gene and oligonucleotide transfer into cells in culture and in vivo: polyethylenimine. Proc Natl Acad Sci USA 92: 7297-7301.
47. Zufferey, R (2002). Production of lentiviral vectors. Current topics in microbiology and immunology 261: 107-121.
48. Eaves, G N, and Jeffries, C D (1963). Isolation and Properties of an Exocellular Nuclease of Serratia Marcescens. Journal of bacteriology 85: 273-278.
49. Bess, J W, Jr., Gorelick, R J, Bosche, W J, Henderson, L E, and Arthur, L O (1997). Microvesicles are a source of contaminating cellular proteins found in purified HIV-1 preparations. Virology 230: 134-144.
50. Segura, M M, Kamen, A, Trudel, P, and Gamier, A (2005). A novel purification strategy for retrovirus gene therapy vectors using heparin affinity chromatography. Biotechnol Bioeng 90: 391-404.
51. Segura, M M, Kamen, A, Lavoie, M C, and Gamier, A (2007). Exploiting heparin-binding properties of MoMLV-based retroviral vectors for affinity chromatography. J Chromatogr B Analyt Technol Biomed Life Sci 846: 124-131.
52. McNally, D J, Darling, D, Farzaneh, F, Levison, P R, and Slater, N K (2014). Optimised concentration and purification of retroviruses using membrane chromatography. J Chromatogr A 1340: 24-32.
53. Whitney, D, McCoy, M, Gordon, N, and Afeyan, N (1998). Characterization of large-pore polymeric supports for use in perfusion biochromatography. J Chromatogr A 807: 165-184.
54. Cruz, P E, Goncalves, D, Almeida, J, Moreira, J L, and Carrondo, M J (2000). Modeling retrovirus production for gene therapy. 2. Integrated optimization of bioreaction and downstream processing. Biotechnol Prog 16: 350-357.
55. Josephson, N C, and Russell, D W (2010). Production of foamy virus vector and transduction of hematopoietic cells. Cold Spring Harb Protoc 2010: pdb prot5481.
56. Kiem, H P, Allen, J, Trobridge, G, Olson, E, Keyser, K, Peterson, L, et al. (2007). Foamy-virus-mediated gene transfer to canine repopulating cells. Blood 109: 65-70.
57. van der Loo, J C, Swaney, W P, Grassman, E, Terwilliger, A, Higashimoto, T, Schambach, A, et al. (2012). Scale-up and manufacturing of clinical-grade self-inactivating gamma-retroviral vectors by transient transfection. Gene Ther 19: 246-254.

What is claimed is:

1. A method of preparing FV vector particles, comprising the steps of:
a. transfecting a population of eukaryotic cells by contacting said population of eukaryotic cells with one or more transfection reagents to form a transfection mixture, and incubating said transfection mixture to form a transfected cell population;
b. harvesting said FV vector particles from said transfected cell population, wherein said harvesting step is carried out about 70 hours to about 100 hours, or about 70 hours to about 90 hours, or about 70 hours to about 80 hours, or about 72 hours to about 75 hours, post-transfection;
c. purifying said FV vector particles;
d. concentrating said FV vector particles.

2. The method of claim 1 wherein said population of eukaryotic cells are pre-seeded for about 20 to about 30 hours, or about 24 hours, prior to said transfecting step.

3. The method of claim 1 wherein said pre-seeding is carried out until said population of eukaryotic cells achieves a cell density of from about 1×10⁵ cells/cm² to about 2×10⁵ cells/cm² or about 1.8×10⁵ cells/cm².

4. The method of claim 1, wherein said pre-seeding step comprises the step of plating eukaryotic cells 1 day prior to PEI transfection with fresh media.

5. The method of claim 1 wherein said pre-seeding step comprises adding poly-L-lysine in an amount sufficient to pre-coat tissue culture plastic with about 3.5 to about 10 mL per 225 cm² surface area, preferably at a concentration of about 0.01%.

6. The method of claim 1 wherein said one or more transfection reagents comprise vector plasmid and a plasmid comprising codon optimized pCiGAGopt.

7. The method of claim 1 wherein said transfecting step occurs in the presence of about 10% (vol/vol) fetal bovine serum and about 0.4% (vol/vol) PEIPro.

8. The method of claim 1, wherein said transfecting step occurs in the presence of about 10% fetal bovine serum and calcium phosphate, butyrate, and chloroquine.

9. The method of claim 1, wherein said transfection mixture is incubated for about 10 to about 20 minutes at ambient temperature (20-24° C.), preferably for about 10 minutes.

10. The method of claim 1, wherein said transfection mixture is maintained in the initial media until the day of harvest.

11. The method of claim 1, wherein the pH of said transfection mixture is less than about 8.

12. The method of claim 1 wherein said FV vector particles is subjected to a filtration step.

13. The method of claim 1 wherein said transfected cell population is contacted with benzonase at a concentration of from about 50 to about 200 U/mL, preferably about 50 U/mL in the presence of about 10 mM MgCl₂ for a period of from about 2 to about 6 hours, preferably about 4 hours prior to a filtration step in one instance, and for 16 to 40 hours prior to vector harvest in another.

14. The method of claim 1 further comprising the step of isolating said FV vector particles using a heparin column.

15. The method of claim 1 further comprising the step of concentrating said FV vector particles using tangential flow filtration.

16. The method of claim 1, further comprising the step of concentrating said FV vector particles, followed by dilution to about 140 mM to about 160 mM, preferably about 150 mM NaCl.

17. The method of claim 1 further comprising the step of concentrating said FV vector particles using tangential flow filtration.

18. The method of claim 1 further comprising the step of concentrating said FV vector particles using ultracentrifugation.

19. The method of claim 1 wherein said FV vector particles are stored at a temperature of about −70° C. to about −90° C., preferably about −80° C. in the presence of DMSO.

20. The method of claim 1 wherein said FV vector particles are stored frozen in the presence of from about 3 to about 5% DMSO, preferably about 5% DMSO.

21. A method of obtaining an increased titer of FV vector particles, comprising the steps of:
a. pre-seeding a population of eukaryotic cells for about 20 to about 30 hours, or about 24 hours, wherein said pre-seeding is carried out until said population of eukaryotic cells achieves a cell density of from about 1×10⁵ cells/cm² to about 2×10⁵ cells/cm² or about 1.8×10⁵ cells/cm²;
b. transfecting a population of eukaryotic cells by contacting said population of eukaryotic cells with one or more transfection reagents, wherein said one or more transfection reagents comprise vector and a plasmid comprising codon optimized pCiGAGopt, wherein said plasmid is used at a concentration of about 0.16 to about 10.4 microgram per 75 cm² culture surface equivalent, preferably 0.65 microgram per 75 cm² culture surface equivalent to form a transfection mixture, and incubating said transfection mixture to form a transfected cell population;
c. harvesting said FV vector particles from said transfected cell population, wherein said harvesting step is carried out about 70 hours to about 100 hours, or about 70 hours to about 90 hours, or about 70 hours to about 80 hours, or about 72 hours to about 75 hours, post-transfection;
d. purifying said FV vector particles, wherein said purification step comprises use of a media comprising heparin;
e. concentrating said FV vector particles;
f. diluting said FV vector particles to about 150 mM NaCl.


SEQUENCE LISTING (7 sequences)

SEQ ID NO: 1 - DNA, 20 bp, Artificial Sequence
  MSCV-F
agtcctccgatagactgcgt

SEQ ID NO: 2 - DNA, 20 bp, Artificial Sequence
  CD18-R
cttcgtgcactcctgagaga

SEQ ID NO: 3 - DNA, 11 bp, Artificial Sequence
  MSCV
tgctgggcctg

SEQ ID NO: 4 - DNA, 20 bp, Artificial Sequence
  HS Albumin -F
gctctcctgcctgttcttta

SEQ ID NO: 5 - DNA, 20 bp, Artificial Sequence
  HS Albumin-R
ggattctgtgcagcatttgg

SEQ ID NO: 6 - DNA, 16 bp, Artificial Sequence
  HS Albumin
tgaaccagttatgtgt

SEQ ID NO: 7 - DNA, 16 bp, Artificial Sequence
  Albumin Gene Specific Taqman Probe
tgaaccagttatgtgt